(19) United States
(12) Patent Application Publication
(10) Pub. No.: US 2020/0181275 A1
(43) Pub. Date: Jun. 11, 2020

(54) COMBINATION THERAPY WITH ICOS AGONIST AND OX40 AGONIST TO TREAT CANCER

(71) Applicant: GLAXOSMITHKLINE INTELLECTUAL PROPERTY DEVELOPMENT LIMITED, BRENTFORD (GB)

(72) Inventors: Christopher B. HOPSON, Collegeville, PA (US); David J. KILIAN, Collegeville, PA (US); Patrick A. MAYES, Devon, PA (US); Sapna YADAYILLI, Collegeville, PA (US); Niranjan YANAMANDRA, Collegeville, PA (US)

(21) Appl. No.: 16/620,782

(22) PCT Filed: Jun. 8, 2018
(86) PCT No.: PCT/IB2018/054169

§ 371 Date: Dec. 9, 2019

Related U.S. Application Data
(60) Provisional application No. 62/517,389, filed on Jun. 9, 2017.

(51) Int. Cl. C07K 16/28; C07K 16/30; A61P 35/00

(52) U.S. Cl. CPC C07K 16/2875; C07K 16/30; A61K 38/00; A61P 35/00; C07K 16/2818

(57) ABSTRACT

The present invention provides methods of treating cancer in a patient in need thereof, the method comprising administering to the patient an effective amount of an agent directed to human ICOS and an effective amount of an agent directed to human OX40 sequentially. The present invention also provides an anti-ICOS antibody or antigen binding fragment thereof and an anti-OX40 antibody or antigen binding fragment thereof for sequential use in treating cancer in a human in need thereof.

### FIELD OF THE INVENTION

The present invention relates generally to immunotherapy in the treatment of human disease. More specifically, the present invention relates to the use of sequenced dosing of immunomodulators such as anti-ICOS antibodies and anti-OX40 antibodies in the treatment of cancer.

### BACKGROUND OF THE INVENTION

Cancer immunity is a multistep process that is tightly regulated by a series of negative immune checkpoint and positive co-stimulatory receptors that when effectively triggered can achieve antitumor response (Mellman, I., et al. (2011) Cancer Immunotherapy Comes of Age. Nature 480(7378), 480-489). However, tumors have established various mechanisms to circumvent immune clearance by altering the responsiveness of the immune infiltrate. In some instances, tumors will be highly dependent on a single mechanism, and in these cases, there is the potential to achieve significant clinical activity with single agent immunomodulatory therapy (Hoos, A. (2016). Development of immuno-oncology drugs—from CTLA4 to PD1 to the next generations. Nat Rev Drug Discov. 15(4), 235-47). However, as tumors often utilize multiple, overlapping and redundant mechanisms to block antitumor immune response, combination therapy will likely be required for durable efficacy across a wide range of tumor types. Therefore, new immune targeted therapies are needed to improve the treatment of all cancers.

Thus, there is a need for combination treatments and strategies for dosing of immunomodulators for the treatment of disease, in particular cancer.

### BRIEF DESCRIPTION OF THE DRAWINGS

FIG. 1 is a set of plots showing anti-ICOS antibody (H2L5 IgG4PE) concentration dependent increase in OX40+ CD4 and CD8 T cells.

FIG. 2 is a set of plots showing anti-ICOS antibody (H2L5 IgG4PE) treatment increased OX40+ CD4 and CD8 T cells in in vitro assays with cancer patient PBMC.

FIG. 3 is a set of plots showing anti-ICOS antibody (H2L5 IgG4PE) treatment increased OX40+ CD4 and CD8 T cells in expanded TIL cultures.

FIG. 4 is set of plots showing anti-OX40 antibody treatment increased ICOS+ CD4 and CD8 T cells in blood while decreasing ICOS+ CD4 in tumors from CT26.

FIG. 5 is set of plots showing anti-ICOS antibody treatment increased OX40+ T cells in blood from CT26 tumor bearing mice.

FIG. 6 is a set of plots showing anti-ICOS antibody treatment increased OX40+ T-reg and CD4 T-effectors in blood from CT26.

FIG. 7 is a set of plots showing anti-ICOS antibody treatment increased OX40+ ICOS− T-cells in tumors from CT26.

FIG. 8 is a set of plots showing changes in OX40+ T cells in blood and spleens from ICOS treated A2058 melanoma tumors in huPBMC model.

FIG. 9 is a table and schematic showing the study design of the anti-ICOS antibody (17G9 clone)/anti-OX40 antibody (OX86 clone) concurrent and phased dosing study described herein.

FIG. 10 is a set of plots showing tumor volume and survival in groups treated with 100 μg anti-ICOS antibody and 100 μg anti-OX40 antibody combination (Group 6), 100 μg anti-OX40 antibody (Group 3), and 100 μs anti-ICOS antibody (Group 4).

FIG. 11 is a set of plots showing tumor volume and survival in groups treated with 10 μs anti-ICOS antibody and 100 μg anti-OX40 antibody combination (Group 7), 100 μg anti-OX40 antibody (Group 3), and 10 μs anti-ICOS antibody (Group 5).

FIG. 12 is a set of plots showing tumor volume and survival of groups treated with phased dosing of anti-ICOS antibody and anti-OX40 antibody with 100 μs anti-OX40 lead in/100 μg anti-ICOS follow up (Group 9), and appropriate controls (Group 8: 100 μs anti-OX40 lead in/100 μs IgG2b follow up; Group 10: 100 μs rat IgG1 lead in/100 μs anti-ICOS follow up).

FIG. 13 is a plot and table showing tumors expressing ICOS and OX40 dual positive T cells.

FIG. 14 is a plot showing further separation of tumors based on regions in TME.

FIGS. 15A-15D are plots showing ICOS and OX40 expression on T-reg and CD8 in tumors. FIG. 15A shows proportions of T regulatory cells expressing ICOS in various tumors. FIG. 15B shows proportions of T regulatory cells expressing OX40 in various tumors. FIG. 15C shows proportions of cytotoxic T cells expressing ICOS in various tumors. FIG. 15D shows proportions of cytotoxic T cells expressing OX40 in various tumors.

FIG. 16: Alignment of the amino acid sequences of 106-222, humanized 106-222 (Hu106), and human acceptor X61012 (GenBank accession number) VH sequences.

FIG. 17: Alignment of the amino acid sequences of 106-222, humanized 106-222 (Hu106), and human acceptor AJ388641 (GenBank accession number) VL sequences.

FIG. 18: Nucleotide sequence of the Hu106 VH gene flanked by SpeI and HindIII sites with the deduced amino acid sequence.

FIG. 19: Nucleotide sequence of the Hu106-222 VL gene flanked by NheI and EcoRI sites with the deduced amino acid sequence.

FIG. 20: Alignment of the amino acid sequences of 119-122, humanized 119-122 (Hu119), and human acceptor Z14189 (GenBank accession number) VH sequences.

FIG. 21: Alignment of the amino acid sequences of 119-122, humanized 119-122 (Hu119), and human acceptor M29469 (GenBank accession number) VL sequences.

FIG. 22: Nucleotide sequence of the Hu119 VH gene flanked by SpeI and HindIII sites with the deduced amino acid sequence.

FIG. 23: Nucleotide sequence of the Hu119 VL gene flanked by NheI and EcoRI sites with the deduced amino acid sequence.

FIG. 24: Nucleotide sequence of mouse 119-43-1 VH cDNA with the deduced amino acid sequence.

FIG. 25: Nucleotide sequence of mouse 119-43-1 VL cDNA and the deduced amino acid sequence.

FIG. 26: Nucleotide sequence of the designed 119-43-1 VH gene flanked by SpeI and HindIII sites with the deduced amino acid sequence.

FIG. 27: Nucleotide sequence of the designed 119-43-1 VL gene flanked by NheI and EcoRI sites with the deduced amino acid sequence.

### SUMMARY OF THE INVENTION

In one aspect, the present invention provides methods of treating cancer in a patient in need thereof comprising administering to the patient an effective amount of an agent directed to human ICOS and an effective amount of an agent directed to human OX40 sequentially. In one embodiment, administration of the agent directed to human ICOS is followed by administration of the agent directed to human OX40. In another embodiment, administration of the agent directed to human OX40 is followed by administration of the agent directed to human ICOS. In one embodiment, the agent directed to human ICOS is an ICOS agonist antibody. In one embodiment, the agent directed to human OX40 is an OX40 agonist antibody.

In one aspect, the present invention provides an anti-ICOS antibody or antigen binding fragment thereof and an anti-OX40 antibody or antigen binding fragment thereof for sequential use in treating cancer in a human in need thereof. In one embodiment administration of the anti-ICOS antibody or antigen binding fragment thereof is followed by administration of the anti-OX40 antibody or antigen binding fragment thereof. In another embodiment administration of the anti-OX40 antibody or antigen binding fragment thereof is followed by administration of the anti-ICOS antibody or antigen binding fragment thereof. In one embodiment, the anti-ICOS antibody is an ICOS agonist antibody. In one embodiment, the anti-OX40 antibody is an OX40 agonist antibody.

### DETAILED DESCRIPTION OF THE INVENTION

### Definitions

As used herein “ICOS” means any Inducible T-cell costimulator protein. Pseudonyms for ICOS (Inducible T-cell COStimulator) include AILIM; CD278; CVID1, JTT-1 or JTT-2, MGC39850, or 8F4. ICOS is a CD28-superfamily costimulatory molecule that is expressed on activated T cells. The protein encoded by this gene belongs to the CD28 and CTLA-4 cell-surface receptor family. It forms homodimers and plays an important role in cell-cell signaling, immune responses, and regulation of cell proliferation. The amino acid sequence of human ICOS (isoform 2) (Accession No.: UniProtKB-Q9Y6W8-2) is shown below as SEQ ID NO:39.

(SEQ ID NO: 39)
MKSGLWYFFLFCLRIKVLTGEINGSANYEMFIFHNGGVQILCKYPDIVQ
QFKMQLLKGGQILCDLTKTKGSGNTVSIKSLKFCHSQLSNNSVSFFLYN
LDHSHANYYFCNLSIFDPPPFKVTLTGGYLHIYESQLCCQLKFWLPIGC
AAFVVVCILGCILICWLTKKM

The amino acid sequence of human ICOS (isoform 1) (Accession No.: UniProtKB - Q9Y6W8-1) is shown below as SEQ ID NO:48.

(SEQ ID NO: 48)
MKSGLWYFFL FCLRIKVLTG EINGSANYEM FIFHNGGVQI
LCKYPDIVQQ FKMQLLKGGQ ILCDLTKTKG SGNTVSIKSL
KFCHSQLSNN SVSFFLYNLD HSHANYYFCN LSIFDPPPFK
VTLTGGYLHI YESQLCCQLK FWLPIGCAAF VVVCILGCIL
ICWLTKKKYS SSVHDPNGEY MFMRAVNTAK KSRLTDVTL

Activation of ICOS occurs through binding by ICOS-L (B7RP-1/B7-H2). Neither B7-1 nor B7-2 (ligands for CD28 and CTLA4) bind or activate ICOS. However, ICOS-L has been shown to bind weakly to both CD28 and CTLA-4 (Yao S et al., “B7-H2 is a costimulatory ligand for CD28 in human”, Immunity, 34(5); 729-40 (2011)). Expression of ICOS appears to be restricted to T cells. ICOS expression levels vary between different T cell subsets and on T cell activation status. ICOS expression has been shown on resting TH17, T follicular helper (TFH) and regulatory T (Treg) cells; however, unlike CD28; it is not highly expressed on naive T_{H}1 and T_{H}2 effector T cell populations (Paulos C M et al., “The inducible costimulator (ICOS) is critical for the development of human Th17 cells”, Sci Transl Med, 2(55); 55ra78 (2010)). ICOS expression is highly induced on CD4+ and CD8+ effector T cells following activation through TCR engagement (Wakamatsu E, et al., “Convergent and divergent effects of costimulatory molecules in conventional and regulatory CD4+ T cells”, Proc Natal Acad Sci USA, 110(3); 1023-8 (2013)). Co-stimulatory signaling through ICOS receptor only occurs in T cells receiving a concurrent TCR activation signal (Sharpe A H and Freeman G J. “The B7-CD28 Superfamily”, Nat. Rev Immunol, 2(2); 116-26 (2002)). In activated antigen specific T cells, ICOS regulates the production of both T_{H}1 and T_{H}2 cytokines including IFN-γ, TNF-α, IL-10, IL-4, IL-13 and others. ICOS also stimulates effector T cell proliferation, albeit to a lesser extent than CD28 (Sharpe A H and Freeman G J. “The B7-CD28 Superfamily”, Nat. Rev Immunol, 2(2); 116-26 (2002)). Antibodies to ICOS and methods of using in the treatment of disease are described, for instance, in WO2012/131004, US20110243929, and US20160215059. US20160215059 is incorporated by reference herein. CDRs for murine antibodies to human ICOS having agonist activity are shown in PCT/EP2012/055735 (WO 2012/131004). Antibodies to ICOS are also disclosed in WO 2008/137915, WO 2010/056804, EP 1374902, EP1374901, and EP1125585. Agonist antibodies to ICOS or ICOS binding proteins are disclosed in WO2012/13004, WO2014/033327, WO2016/120789, US20160215059, and US20160304610. Exemplary antibodies in US2016/0304610 include 37A10S713. Sequences of 37A10S713 are reproduced below as SEQ ID NOS: 49-56.

### 37A105713 Heavy Chain Variable Region:

(SEQ. ID NO: 49)
EVQLVESGG LVQPGGSLRL SCAASGFTFS DYWMDWVRQA
PGKGLVWVSN IDEDGSITEY SPFVKGRFTI SRDNAKNTLY
LQMNSLRAED TAVYYCTRWG RFGFDSWGQG TLVTVSS

### 37A10S713 Light Chain Variable Region:

(SEQ. ID NO: 50)
DIVMTQSPDS LAVSLGERAT INCKSSQSLL SGSFNYLTWY
QQKPGQPPKL LIFYASTRHT GVPDRFSGSG SGTDFTLTIS
SLQAEDVAVY YCHHHYNAPP TFGPGTKVDI K
37A10S713 V_{H} CDR1:
(SEQ.ID NO: 51)
GFTFSDYWMD
37A10S713 V_{H} CDR2:
(SEQ. ID NO: 52)
NIDEDGSITEYSPFVKG
37A10S713 V_{H} CDR3:
(SEQ. ID. NO: 53)
WGRFGFDS
37A10S713 V_{L} CDR1:
(SEQ. ID NO: 54)
KSSQSLLSGSFNYLT
37A10S713 V_{L} CDR2:
(SEQ. ID NO: 55)
YASTRHT
37A10S713 V_{L} CDR3:
(SEQ. ID NO: 56)
HHHYNAPPT

By “agent directed to ICOS” is meant any chemical compound or biological molecule capable of binding to ICOS. In some embodiments, the agent directed to ICOS is an ICOS binding protein. In some other embodiments, the agent directed to ICOS is an ICOS agonist.

The term “ICOS binding protein” as used herein refers to antibodies and other protein constructs, such as domains, which are capable of binding to ICOS. In some instances, the ICOS is human ICOS. The term “ICOS binding protein” can be used interchangeably with “ICOS antigen binding protein.” Thus, as is understood in the art, anti-ICOS antibodies and/or ICOS antigen binding proteins would be considered ICOS binding proteins. As used herein, “antigen binding protein” is any protein, including but not limited to antibodies, domains and other constructs described herein, that binds to an antigen, such as ICOS. As used herein “antigen binding portion” of an ICOS binding protein would include any portion of the ICOS binding protein capable of binding to ICOS, including but not limited to, an antigen binding antibody fragment.

In one embodiment, the ICOS antibodies of the present invention comprise any one or a combination of the following CDRs:

CDRH1:
(SEQ ID NO: 40)
DYAMH
CDRH2:
(SEQ ID NO: 41)
LISIYSDHTNYNQKFQG
CDRH3:
(SEQ ID NO: 42)
NNYGNYGWYFDV
CDRL1:
(SEQ ID NO: 43)
SASSSVSYMH
CDRL2:
(SEQ ID NO: 44)
DTSKLAS
CDRL3:
(SEQ ID NO: 45)
FQGSGYPYT

In some embodiments, the anti-ICOS antibodies of the present invention comprise a heavy chain variable region having at least 90% sequence identity to SEQ ID NO:46. Suitably, the ICOS binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:46.

### Humanized Heavy Chain (V_{H}) Variable Region (H2):

(SEQ ID NO: 46)
QVQLVQSGAE VKKPGSSVKV SCKASGYTFT DYAMHWVRQA
PGQGLEWMGL ISIYSDHTNY NQKFQGRVTI TADKSTSTAY
MELSSLRSED TAVYYCGRNN YGNYGWYFDV WGQGTTVTVS S

In one embodiment of the present invention the ICOS antibody comprises CDRL1 (SEQ ID NO:43), CDRL2 (SEQ ID NO:44), and CDRL3 (SEQ ID NO:45) in the light chain variable region having the amino acid sequence set forth in SEQ ID NO:47. ICOS binding proteins of the present invention comprising the humanized light chain variable region set forth in SEQ ID NO:47 are designated as “L5.” Thus, an ICOS binding protein of the present invention comprising the heavy chain variable region of SEQ ID NO:46 and the light chain variable region of SEQ ID NO:47 can be designated as H2L5 herein.

In some embodiments, the ICOS binding proteins of the present invention comprise a light chain variable region having at least 90% sequence identity to the amino acid sequence set forth in SEQ ID NO:47. Suitably, the ICOS binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:47.

### Humanized Light Chain (V_{L}) Variable Region (L5)

(SEQ ID NO: 47)
EIVLTQSPAT LSLSPGERAT LSCSASSSVS YMHWYQQKPG
QAPRLLIYDT SKLASGIPAR FSGSGSGTDY TLTISSLEPE
DFAVYYCFQG SGYPYTFGQG TKLEIK

CDRs or minimum binding units may be modified by at least one amino acid substitution, deletion or addition, wherein the variant antigen binding protein substantially retains the biological characteristics of the unmodified protein, such as an antibody comprising SEQ ID NO:46 and SEQ ID NO:47.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Table 1 below.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

The subclass of an antibody in part determines secondary effector functions, such as complement activation or Fc receptor (FcR) binding and antibody dependent cell cytotoxicity (ADCC) (Huber, et al., Nature 229(5284): 419-20 (1971); Brunhouse, et al., Mol Immunol 16(11): 907-17 (1979)). In identifying the optimal type of antibody for a particular application, the effector functions of the antibodies can be taken into account. For example, hIgG1 antibodies have a relatively long half life, are very effective at fixing complement, and they bind to both FcγRI and FcγRII. In contrast, human IgG4 antibodies have a shorter half life, do not fix complement and have a lower affinity for the FcRs. Replacement of serine 228 with a proline (S228P) in the Fc region of IgG4 reduces heterogeneity observed with hIgG4 and extends the serum half life (Kabat, et al., “Sequences of proteins of immunological interest” 5.sup.th Edition (1991); Angal, et al., Mol Immunol 30(1): 105-8 (1993)). A second mutation that replaces leucine 235 with a glutamic acid (L235E) eliminates the residual FcR binding and complement binding activities (Alegre, et al., J Immunol 148(11): 3461-8 (1992)). The resulting antibody with both mutations is referred to as IgG4PE. The numbering of the hIgG4 amino acids was derived from EU numbering reference: Edelman, G M et al., Proc. Natl. Acad. USA, 63, 78-85 (1969). PMID: 5257969. In one embodiment of the present invention the ICOS antibody is an IgG4 isotype. In one embodiment, the ICOS antibody comprises an IgG4 Fc region comprising the replacement S228P and L235E may have the designation IgG4PE. In one embodiment, the ICOS antibody is H2L5 IgG4PE.

As used herein “ICOS-L” and “ICOS Ligand” are used interchangeably and refer to the membrane bound natural ligand of human ICOS. ICOS ligand is a protein that in humans is encoded by the ICOSLG gene. ICOSLG has also been designated as CD275 (cluster of differentiation 275). Pseudonyms for ICOS-L include B7RP-1 and B7-H2.

As used herein, an “agent directed to OX40” or “agent directed to OX-40” means any chemical compound or biological molecule capable of binding to OX40. In some embodiments, the agent directed to OX40 is an OX40 agonist. In some embodiments, the agent directed to OX40 is an OX40 binding protein.

The term “OX40 binding protein” as used herein refers to antibodies and other protein constructs, such as domains, which are capable of binding to OX40. In some instances, the OX40 is human OX40. The term “OX40 binding protein” can be used interchangeably with “OX40 antigen binding protein.” Thus, as is understood in the art, anti-OX40 antibodies and/or OX40 antigen binding proteins would be considered OX40 binding proteins. As used herein, “antigen binding protein” is any protein, including but not limited to antibodies, domains and other constructs described herein, that binds to an antigen, such as OX40. As used herein “antigen binding portion” of an OX40 binding protein would include any portion of the OX40 binding protein capable of binding to OX40, including but not limited to, an antigen binding antibody fragment.

CD134, also known as OX40, is a member of the TNFR-superfamily of receptors which is not constitutively expressed on resting naïve T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. OX40/OX40-ligand (OX40 Receptor)/(OX40L) are a pair of costimulatory molecules critical for T cell proliferation, survival, cytokine production, and memory cell generation. Early in vitro experiments demonstrated that signaling through OX40 on CD4⁺ T cells lead to TH2, but not TH1 development. These results were supported by in vivo studies showing that blocking OX40/OX40L interaction prevented the induction and maintenance of TH2-mediated allergic immune responses. However, blocking OX40/OX40L interaction ameliorates or prevents TH1-mediated diseases. Furthermore, administration of soluble OX40L or gene transfer of OX40L into tumors were shown to strongly enhance anti-tumor immunity in mice. Recent studies also suggest that OX40/OX40L may play a role in promoting CD8 T cell-mediated immune responses. As discussed herein, OX40 signaling blocks the inhibitory function of CD4⁺ CD25⁺ naturally occurring regulatory T cells and the OX40/OX40L pair plays a critical role in the global regulation of peripheral immunity versus tolerance. OX-40 antibodies, OX-40 fusion proteins and methods of using them are disclosed in US Patent Nos: U.S. Pat. Nos. 7,504,101; 7,758,852; 7,858,765; 7,550,140; 7,960,515; and 9,006,399 and international publications: WO 2003082919; WO 2003068819; WO 2006063067; WO 2007084559; WO 2008051424; WO2012027328; and WO2013028231.

Herein an antigen binding protein (ABP) of the invention or an anti-OX40 antigen binding protein is one that binds OX40, and in some embodiments, does one or more of the following: modulate signaling through OX40, modulates the function of OX40, agonize OX40 signaling, stimulate OX40 function, or co-stimulate OX40 signaling Example 1 of U.S. Pat. No. 9,006,399 discloses an OX40 binding assay. One of skill in the art would readily recognize a variety of other well known assays to establish such functions.

In one embodiment, the OX40 antigen binding protein is one disclosed in WO2012/027328 (PCT/US2011/048752), international filing date 23 Aug. 2011. In another embodiment, the antigen binding protein comprises the CDRs of an antibody disclosed in WO2012/027328 (PCT/US2011/048752), international filing date 23 Aug. 2011, or CDRs with 90% identity to the disclosed CDR sequences. In a further embodiment the antigen binding protein comprises a VH, a VL, or both of an antibody disclosed in WO2012/027328 (PCT/US2011/048752), international filing date 23 Aug. 2011, or a VH or a VL with 90% identity to the disclosed VH or VL sequences.

In another embodiment, the OX40 antigen binding protein is disclosed in WO02013/028231 (PCT/US2012/024570), international filing date 9 Feb. 2012. In another embodiment, the antigen binding protein comprises the CDRs of an antibody disclosed in WO2013/028231 (PCT/US2012/024570), international filing date 9 Feb. 2012, or CDRs with 90% identity to the disclosed CDR sequences. In a further embodiment, the antigen binding protein comprises a VH, a VL, or both of an antibody disclosed in WO2013/028231 (PCT/US2012/024570), international filing date 9 Feb. 2012, or a VH or a VL with 90% identity to the disclosed VH or VL sequences.

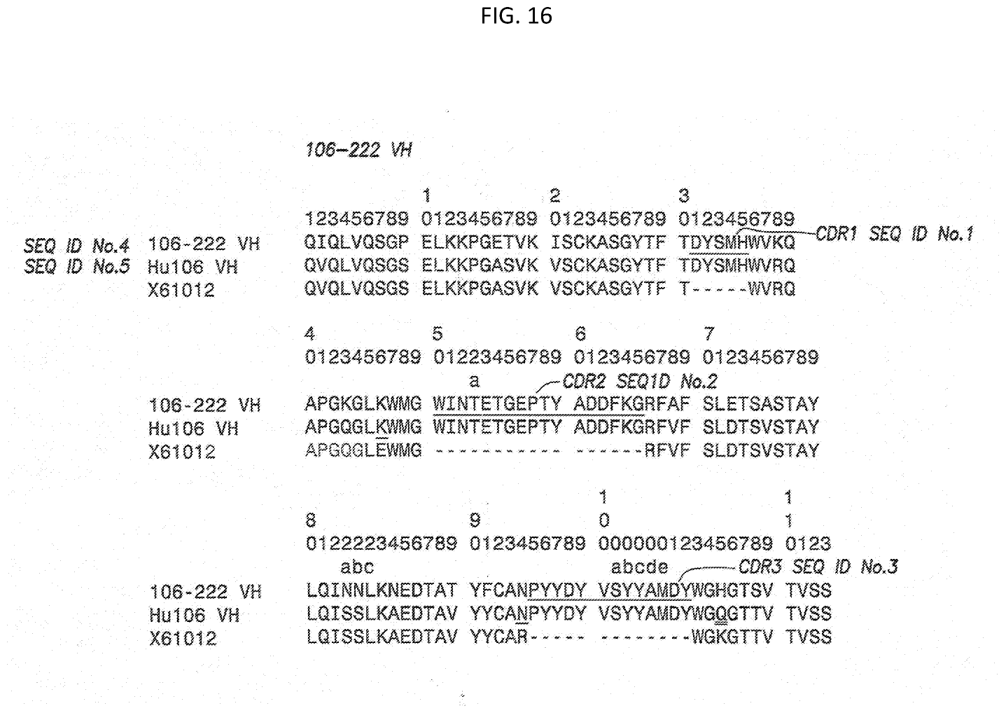

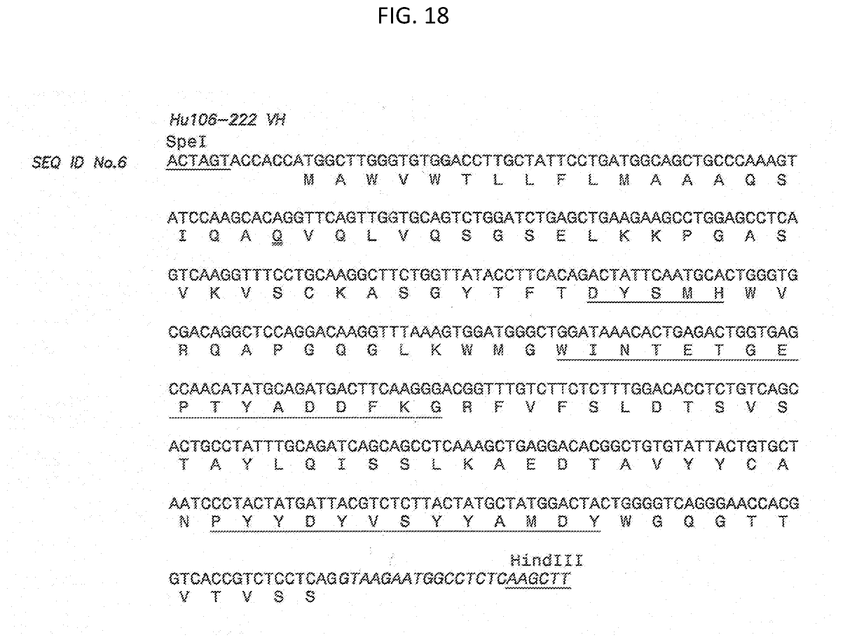

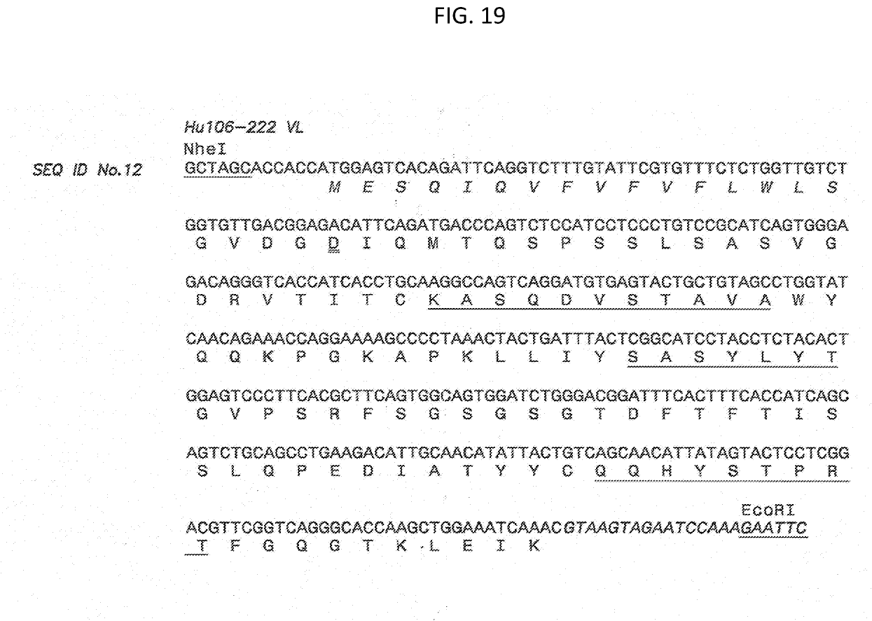

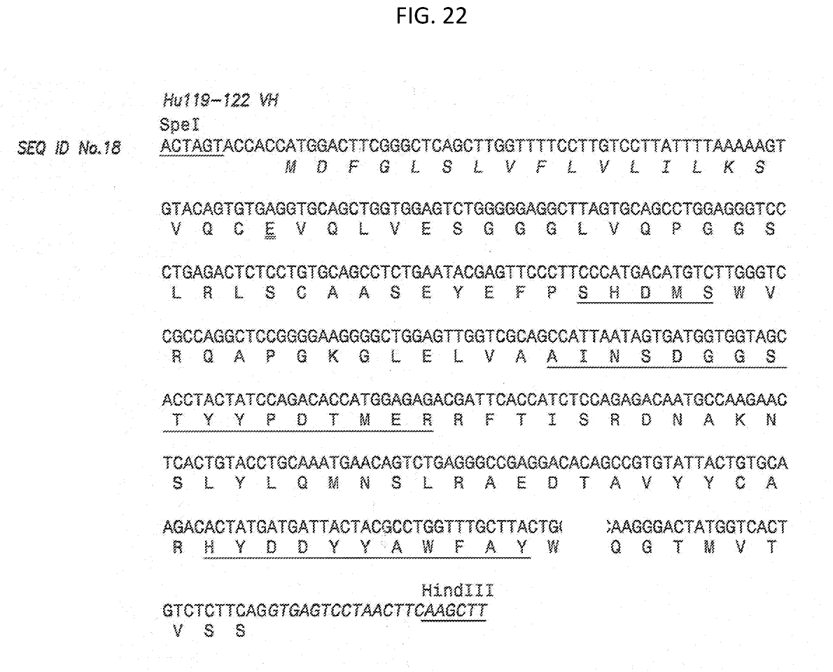

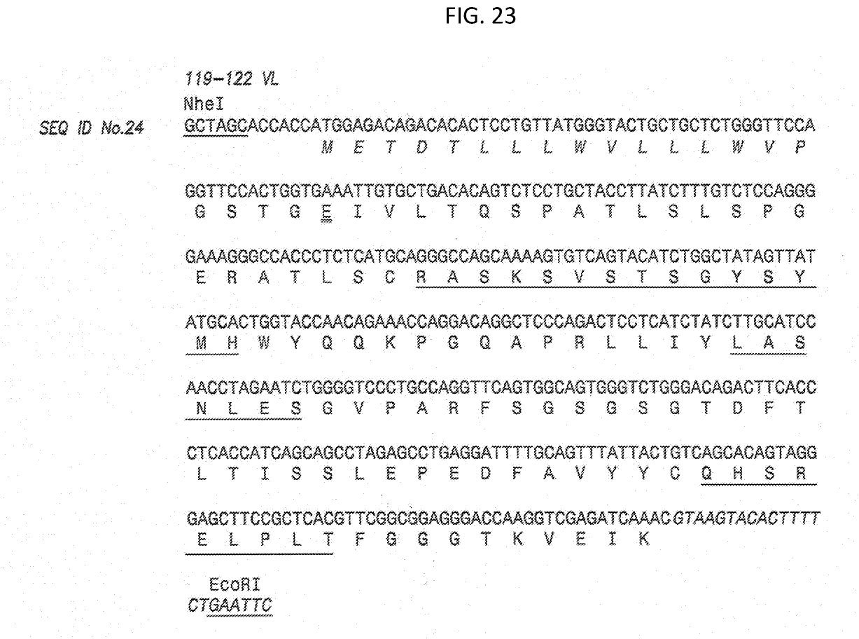

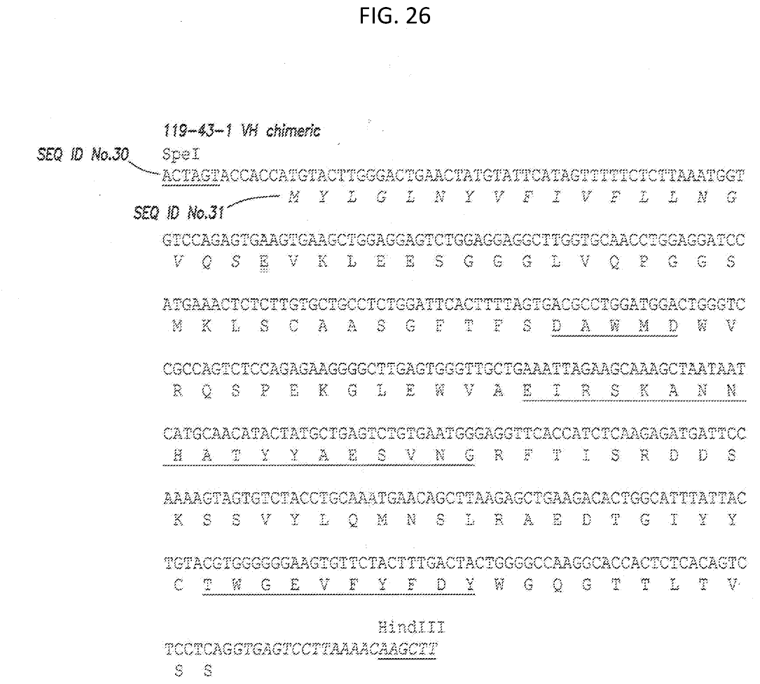

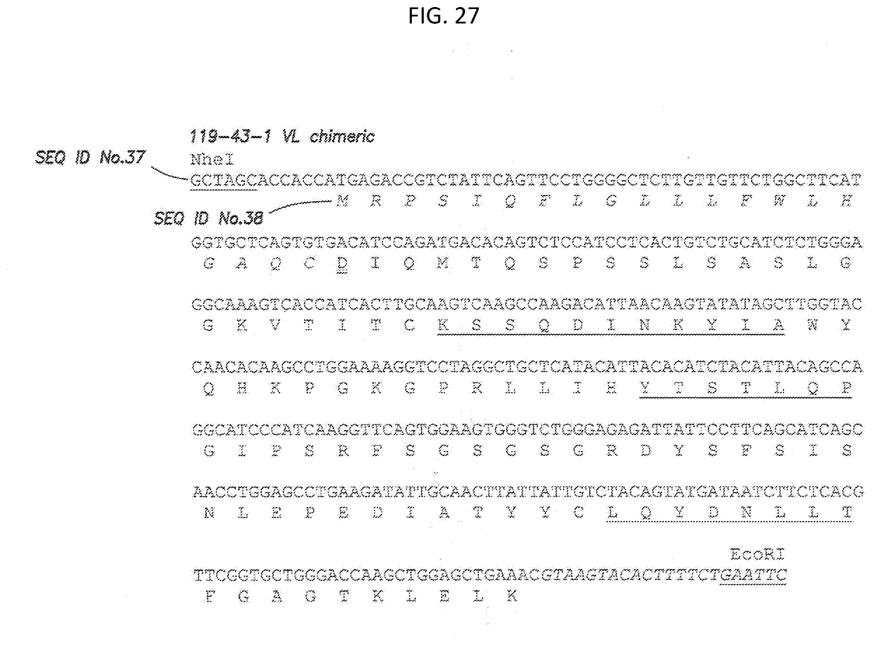

In another embodiment, the anti-OX40 ABP or antibody of the invention comprises one or more of the CDRs or VH or VL sequences, or sequences with 90% identity thereto, shown in FIGS. 16 to 27 herein.

In one embodiment, the anti-OX40 ABP or antibody of the present invention comprises any one or a combination of the following CDRs:

CDRH1:
(SEQ ID NO: 1)
DYSMH
CDRH2:
(SEQ ID NO: 2)
WINTETGEPTYADDFKG
CDRH3:
(SEQ ID NO: 3)
PYYDYVSYYAMDY
CDRL1:
(SEQ ID NO: 7)
KASQDVSTAVA
CDRL2:
(SEQ ID NO: 8)
SASYLYT
CDRL3:
(SEQ ID NO: 9)
QQHYSTPRT

In some embodiments, the anti-OX40 ABP or antibodies of the present invention comprise a heavy chain variable region having at least 90% sequence identity to SEQ ID NO:5. Suitably, the 0X40 binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:5.

### Humanized Heavy Chain (V_{H}) Variable Region:

(SEQ ID NO: 5)
QVQLVQSGS ELKKPGASVK VSCKASGYTF TDYSMHWVRQ
APGQGLKWMG WINTETGEPTY ADDFKGRFVF SLDTSVSTAY
LQISSLKAEDTAV YYCANPYYDY VSYYAMDYWGQGTTV TVSS

In one embodiment of the present invention the OX40 ABP or antibody comprises CDRL1 (SEQ ID NO:7), CDRL2 (SEQ ID NO:8), and CDRL3 (SEQ ID NO:9) in the light chain variable region having the amino acid sequence set forth in SEQ ID NO:11. In some embodiments, 0X40 binding proteins of the present invention comprise the light chain variable region set forth in SEQ ID NO:11. In one embodiment, an 0X40 binding protein of the present invention comprises the heavy chain variable region of SEQ ID NO:5 and the light chain variable region of SEQ ID NO:11.

### Humanized Light Chain (V_{L}) Variable Region

(SEQ ID NO: 11)
DIQMTQSPS SLSASVGDRV TITCKASQDV STAVAWYQQK
PGKAPKLLIY SASYLYTGVP SRFSGSGSGT DFTFTISSLQ
PEDIATYYCQ QHYSTPRTFG QGTKLEIK

In some embodiments, the OX40 binding proteins of the present invention comprise a light chain variable region having at least 90% sequence identity to the amino acid sequence set forth in SEQ ID NO:11. Suitably, the OX40 binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:11.

In another embodiment, the anti-0X40 ABP or antibody of the present invention comprise any one or a combination of the following CDRs:

CDRH1:
(SEQ ID NO: 13)
SHDMS
CDRH2:
(SEQ ID NO: 14)
AINSDGGSTYYPDTMER
CDRH3:
(SEQ ID NO: 15)
HYDDYYAWFAY
CDRL1:
(SEQ ID NO: 19)
RASKSVSTSGYSYMH
CDRL2:
(SEQ ID NO: 20)
LASNLES
CDRL3:
(SEQ ID NO: 21)
QHSRELPLT

In some embodiments, the anti-OX40 ABP or antibodies of the present invention comprise a heavy chain variable region having at least 90% sequence identity to SEQ ID NO:17. Suitably, the OX40 binding proteins of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:17.

### Humanized Heavy Chain (V_{H}) Variable Region:

(SEQ ID NO: 17)
EVQLVESGG GLVQPGGSLR LSCAASEYEF PSHDMSWVRQ
APGKGLELVA AINSDGGSTYY PDTMERRFTI SRDNAKNSLY
LQMNSLRAEDTAV YYCARHYDDY YAWFAYWGQGTMV TVSS

In one embodiment of the present invention the OX40 ABP or antibody comprises CDRL1 (SEQ ID NO:19), CDRL2 (SEQ ID NO:20), and CDRL3 (SEQ ID NO:21) in the light chain variable region having the amino acid sequence set forth in SEQ ID NO:23. In some embodiments, OX40 binding proteins of the present invention comprise the light chain variable region set forth in SEQ ID NO:23. In one embodiment, an OX40 binding protein of the present invention comprises the heavy chain variable region of SEQ ID NO:17 and the light chain variable region of SEQ ID NO:23.

### Humanized Light Chain (V_{L}) Variable Region

(SEQ ID NO: 23)
EIVLTQSPA TLSLSPGERA TLSCRASKSVSTSG YSYMHWYQQK
PGQAPRLLIY LASNLESGVP ARFSGSGSGT DFTLTISSLE
PEDFAVYYCQ HSRELPLTFG GGTKVEIK

In some embodiments, the OX40 binding proteins of the present invention comprise a light chain variable region having at least 90% sequence identity to the amino acid sequence set forth in SEQ ID NO:23. Suitably, the OX40 binding proteins of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:23.

CDRs or minimum binding units may be modified by at least one amino acid substitution, deletion or addition, wherein the variant antigen binding protein substantially retains the biological characteristics of the unmodified protein, such as an antibody comprising SEQ ID NO:5 and SEQ ID NO:11 or an antibody comprising SEQ ID NO: 17 and SEQ ID NO: 23.

It will be appreciated that each of CDR H1, H2, H3, L1, L2, L3 may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Table 1.

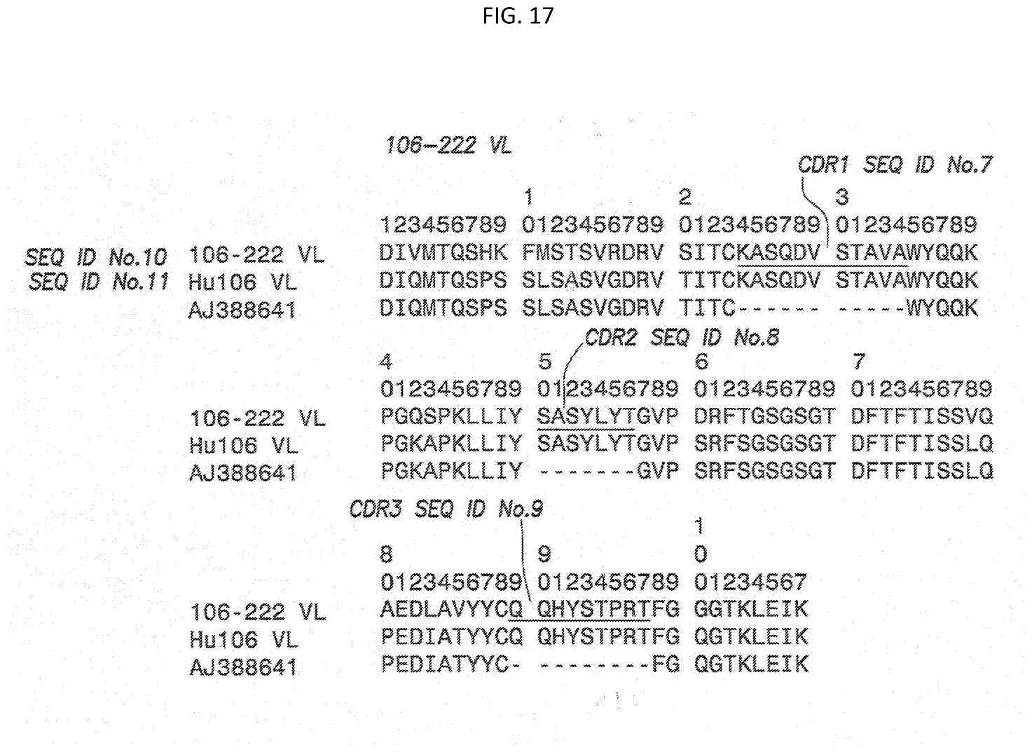

In one embodiment, the ABP or antibody of the invention comprises the CDRs of the 106-222 antibody, e.g., of FIGS. 16-17 herein, e.g., CDRH1, CDRH2, and CDRH3 having the amino acid sequence as set forth in SEQ ID NOs 1, 2, and 3, as disclosed in FIG. 16, and e.g., CDRL1, CDRL2, and CDRL3 having the sequences as set forth in SEQ ID NOs 7, 8, and 9 respectively. In one embodiment, the ABP or antibody of the invention comprises the CDRs of the 106-222, Hu106 or Hu106-222 antibody as disclosed in WO2012/027328 (PCT/US2011/048752), international filing date 23 Aug. 2011. In a further embodiment, the anti-OX40 ABP or antibody of the invention comprises the VH and VL regions of the 106-222 antibody as shown in FIGS. 16-17 herein, e.g., a VH having an amino acid sequence as set forth in SEQ ID NO:4 and a VL as in FIG. 17 having an amino acid sequence as set forth in SEQ ID NO: 10. In another embodiment, the ABP or antibody of the invention comprises a VH having an amino acid sequence as set forth in SEQ ID NO: 5 in FIG. 16 herein, and a VL having an amino acid sequence as set forth in SEQ ID NO:11 in FIG. 17 herein. In a further embodiment, the anti-OX40 ABP or antibody of the invention comprises the VH and VL regions of the Hu106-222 antibody or the 106-222 antibody or the Hu106 antibody as disclosed in WO2012/027328 (PCT/US2011/048752), international filing date 23 Aug. 2011. In a further embodiment, the anti-OX40 ABP or antibody of the invention is 106-222, Hu106-222 or Hu106, e.g., as disclosed in WO2012/027328 (PCT/US2011/048752), international filing date 23 Aug. 2011. In a further embodiment, the ABP or antibody of the invention comprises CDRs or VH or VL or antibody sequences with 90% identity to the sequences in this paragraph.

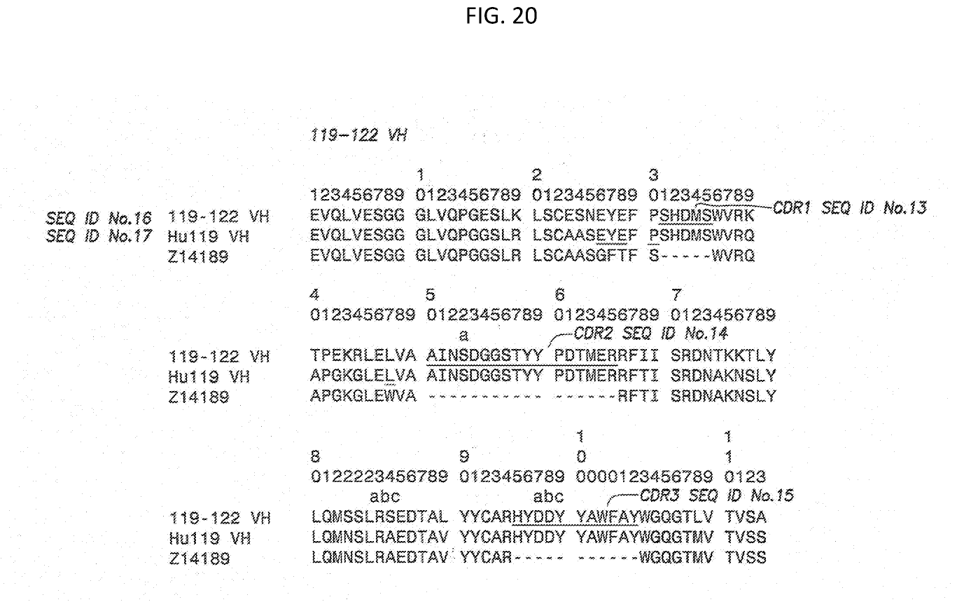

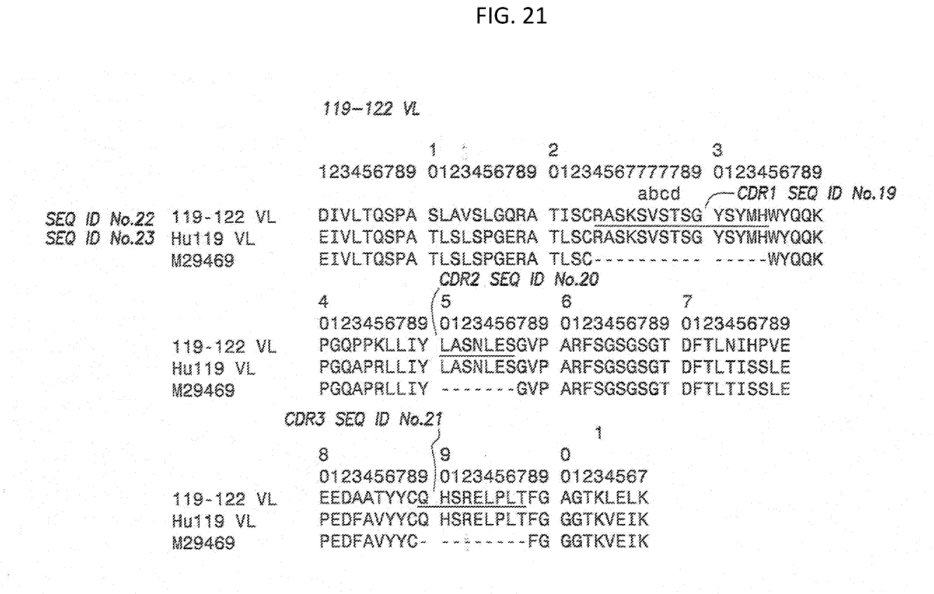

In another embodiment, the anti-OX40 ABP or antibody of the invention comprises the CDRs of the 119-122 antibody, e.g., of FIGS. 20-21 herein, e.g., CDRH1, CDRH2, and CDRH3 having the amino acid sequence as set forth in SEQ ID NOs 13, 14, and 15 respectively . In another embodiment, the anti-OX40 ABP or antibody of the invention comprises the CDRs of the 119-122 or Hu119 or Hu119-222 antibody as disclosed in WO2012/027328 (PCT/US2011/048752), international filing date 23 Aug. 2011. In a further embodiment, the anti-OX40 ABP or antibody of the invention comprises a VH having an amino acid sequence as set forth in SEQ ID NO: 16 in FIG. 20 herein, and a VL having the amino acid sequence as set forth in SEQ ID NO: 22 as shown in FIG. 21 herein. In another embodiment, the anti-OX40 ABP or antibody of the invention comprises a VH having an amino acid sequence as set forth in SEQ ID NO: 17 and a VL having the amino acid sequence as set forth in SEQ ID NO: 23. In a further embodiment, the anti-OX40 ABP or antibody of the invention comprises the VH and VL regions of the 119-122 or Hu119 or Hu119-222 antibody as disclosed in WO2012/027328 (PCT/US2011/048752), international filing date 23 Aug. 2011. In a further embodiment, the ABP or antibody of the invention is 119-222 or Hu119 or Hu119-222 antibody, e.g., as disclosed in WO2012/027328 (PCT/US2011/048752), international filing date 23 Aug. 2011. In a further embodiment, the ABP or antibody of the invention comprises CDRs or VH or VL or antibody sequences with 90% identity to the sequences in this paragraph.

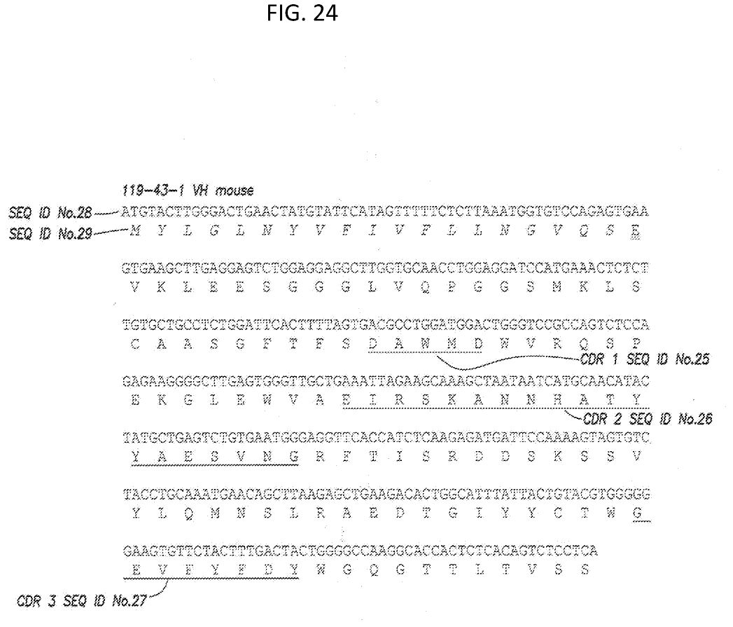

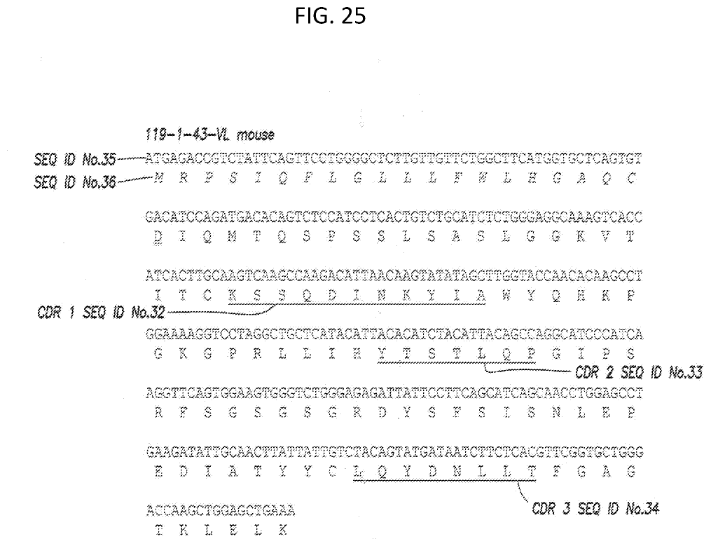

In another embodiment, the anti-OX40 ABP or antibody of the invention comprises the CDRs of the 119-43-1 antibody, e.g., as shown in FIGS. 24-25 herein. In another embodiment, the anti-OX40 ABP or antibody of the invention comprises the CDRs of the 119-43-1 antibody as disclosed in WO2013/028231 (PCT/US2012/024570), international filing date 9 Feb. 2012. In a further embodiment, the anti-OX40 ABP or antibody of the invention comprises one of the VH and one of the VL regions of the 119-43-1 antibody as shown in FIGS. 24-27. In a further embodiment, the anti-OX40 ABP or antibody of the invention comprises the VH and VL regions of the 119-43-1 antibody as disclosed in WO2013/028231 (PCT/US2012/024570), international filing date 9 Feb. 2012. In a further embodiment, the ABP or antibody of the invention is 119-43-1 or 119-43-1 chimeric as disclosed in FIGS. 24-27 herein. In a further embodiment, the ABP or antibody of the invention as disclosed in WO2013/028231 (PCT/US2012/024570), international filing date 9 Feb. 2012. In further embodiments, any one of the ABPs or antibodies described in this paragraph are humanized In further embodiments, any one of the any one of the ABPs or antibodies described in this paragraph are engineered to make a humanized antibody. In a further embodiment, the ABP or antibody of the invention comprises CDRs or VH or VL or antibody sequences with 90% identity to the sequences in this paragraph.

In another embodiment, any mouse or chimeric sequences of any anti-OX40 ABP or antibody of the invention are engineered to make a humanized antibody.

In one embodiment, the anti-OX40 ABP or antibody of the invention comprises: (a) a heavy chain variable region CDR1 comprising the amino acid sequence of SEQ ID NO: 1; (b) a heavy chain variable region CDR2 comprising the amino acid sequence of SEQ ID NO: 2; (c) a heavy chain variable region CDR3 comprising the amino acid sequence of SEQ ID NO. 3; (d) a light chain variable region CDR1 comprising the amino acid sequence of SEQ ID NO. 7; (e) a light chain variable region CDR2 comprising the amino acid sequence of SEQ ID NO. 8; and (f) a light chain variable region CDR3 comprising the amino acid sequence of SEQ ID NO. 9.

In another embodiment, the anti-OX40 ABP or antibody of the invention comprises: (a) a heavy chain variable region CDR1 comprising the amino acid sequence of SEQ ID NO: 13; (b) a heavy chain variable region CDR2 comprising the amino acid sequence of SEQ ID NO: 14; (c) a heavy chain variable region CDR3 comprising the amino acid sequence of SEQ ID NO. 15; (d) a light chain variable region CDR1 comprising the amino acid sequence of SEQ ID NO. 19; (e) a light chain variable region CDR2 comprising the amino acid sequence of SEQ ID NO. 20; and (f) a light chain variable region CDR3 comprising the amino acid sequence of SEQ ID NO. 21.

In another embodiment, the anti-OX40 ABP or antibody of the invention comprises: a heavy chain variable region CDR1 comprising the amino acid sequence of SEQ ID NO: 1 or 13; a heavy chain variable region CDR2 comprising the amino acid sequence of SEQ ID NO: 2 or 14; and/or a heavy chain variable region CDR3 comprising the amino acid sequence of SEQ ID NO: 3 or 15, or a heavy chain variable region CDR having 90% identity thereto.

In yet another embodiment, the anti-OX40 ABP or antibody of the invention comprises: a light chain variable region CDR1 comprising the amino acid sequence of SEQ ID NO: 7 or 19; a light chain variable region CDR2 comprising the amino acid sequence of SEQ ID NO: 8 or 20 and/or a light chain variable region CDR3 comprising the amino acid sequence of SEQ ID NO: 9 or 21, or a heavy chain variable region having 90 percent identity thereto.

In a further embodiment, the anti-OX40 ABP or antibody of the invention comprises: a light chain variable region (“VL”) comprising the amino acid sequence of SEQ ID NO: 10, 11, 22 or 23, or an amino acid sequence with at least 90 percent identity to the amino acid sequences of SEQ ID NO: 10, 11, 22 or 23. In another embodiment, the anti-OX40 ABP or antibody of the invention comprises a heavy chain variable region (“VH”) comprising the amino acid sequence of SEQ ID NO: 4, 5, 16 and 17, or an amino acid sequence with at least 90 percent identity to the amino acid sequences of SEQ ID NO: 4, 5, 16 and 17. In another embodiment, the anti-OX40 ABP or antibody of the invention comprises a variable heavy chain sequence of SEQ ID NO:5 and a variable light chain sequence of SEQ ID NO: 11, or a sequence having 90 percent identity thereto. In another embodiment, the anti-OX40 ABP or antibody of the invention comprises a variable heavy chain sequence of SEQ ID NO:17 and a variable light chain sequence of SEQ ID NO: 23 or a sequence having 90 percent identity thereto.

In another embodiment, the anti-OX40 ABP or antibody of the invention comprises a variable light chain encoded by the nucleic acid sequence of SEQ ID NO: 12, or 24, or a nucleic acid sequence with at least 90 percent identity to the nucleotide sequences of SEQ ID NO: 12 or 24. In another embodiment, the anti-OX40 ABP or antibody of the invention comprises a variable heavy chain encoded by a nucleic acid sequence of SEQ ID NO: 6 or 18, or a nucleic acid sequence with at least 90 percent identity to nucleotide sequences of SEQ ID NO: 6 or 18.

Also provided herein are monoclonal antibodies. In one embodiment, the monoclonal antibodies comprise a variable light chain comprising the amino acid sequence of SEQ ID NO: 10 or 22, or an amino acid sequence with at least 90 percent identity to the amino acid sequences of SEQ ID NO: 10 or 22. Further provided are monoclonal antibodies comprising a variable heavy chain comprising the amino acid sequence of SEQ ID NO: 4 or 16, or an amino acid sequence with at least 90 percent identity to the amino acid sequences of SEQ ID NO: 4 or 16.

As used herein the term “agonist” refers to an antigen binding protein including but not limited to an antibody, which upon contact with a co-signaling receptor causes one or more of the following (1) stimulates or activates the receptor, (2) enhances, increases or promotes, induces or prolongs an activity, function or presence of the receptor and/or (3) enhances, increases, promotes or induces the expression of the receptor. Agonist activity can be measured in vitro by various assays know in the art such as, but not limited to, measurement of cell signaling, cell proliferation, immune cell activation markers, cytokine production. Agonist activity can also be measured in vivo by various assays that measure surrogate end points such as, but not limited to the measurement of T cell proliferation or cytokine production.

As used herein the term “antagonist” refers to an antigen binding protein including but not limited to an antibody, which upon contact with a co-signaling receptor causes one or more of the following (1) attenuates, blocks or inactivates the receptor and/or blocks activation of a receptor by its natural ligand, (2) reduces, decreases or shortens the activity, function or presence of the receptor and/or (3) reduces, descrease, abrogates the expression of the receptor. Antagonist activity can be measured in vitro by various assays know in the art such as, but not limited to, measurement of an increase or decrease in cell signaling, cell proliferation, immune cell activation markers, cytokine production. Antagonist activity can also be measured in vivo by various assays that measure surrogate end points such as, but not limited to the measurement of T cell proliferation or cytokine production.

As used herein the term “cross competes for binding” refers to any agent such as an antibody that will compete for binding to a target with any of the agents of the present invention. Competition for binding between two antibodies can be tested by various methods known in the art including Flow cytometry, Meso Scale Discovery and ELISA. Binding can be measured directly, meaning two or more binding proteins can be put in contact with a co-signaling receptor and bind may be measured for one or each. Alternatively, binding of molecules or interest can be tested against the binding or natural ligand and quantitatively compared with each other.

The term “binding protein” as used herein refers to antibodies and other protein constructs, such as domains, which are capable of binding to an antigen.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanized, multispecific antibodies, including bispecific antibodies, and heteroconjugate antibodies; a single variable domain (e.g., V_{H}, V_{HH}, VL, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer or an EGF domain.

The term “domain” refers to a folded protein structure which retains its tertiary structure independent of the rest of the protein. Generally domains are responsible for discrete functional properties of proteins and in many cases may be added, removed or transferred to other proteins without loss of function of the remainder of the protein and/or of the domain.

The term “single variable domain” refers to a folded polypeptide domain comprising sequences characteristic of antibody variable domains It therefore includes complete antibody variable domains such as V_{H}, V_{HH} and V_{L} and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain A single variable domain is capable of binding an antigen or epitope independently of a different variable region or domain A “domain antibody” or “dAb™” may be considered the same as a “single variable domain” A single variable domain may be a human single variable domain, but also includes single variable domains from other species such as rodent nurse shark and Camelid V_{HH} dAbs™. Camelid V_{HH} are immunoglobulin single variable domain polypeptides that are derived from species including camel, llama, alpaca, dromedary, and guanaco, which produce heavy chain antibodies naturally devoid of light chains Such V_{HH} domains may be humanized according to standard techniques available in the art, and such domains are considered to be “single variable domains” As used herein V_{H} includes camelid V_{HH} domains.

An antigen binding fragment may be provided by means of arrangement of one or more CDRs on non-antibody protein scaffolds. “Protein Scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example an IgG, or IgA scaffold. The IgG scaffold may comprise some or all the domains of an antibody (i.e. CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

Affinity is the strength of binding of one molecule, e.g. an antigen binding protein of the invention, to another, e.g. its target antigen, at a single binding site. The binding affinity of an antigen binding protein to its target may be determined by equilibrium methods (e.g. enzyme-linked immunoabsorbent assay (ELISA) or radioimmunoassay (RIA)), or kinetics (e.g. BIACORE™ analysis). For example, the Biacore™ methods described in Example 5 may be used to measure binding affinity.

Avidity is the sum total of the strength of binding of two molecules to one another at multiple sites, e.g. taking into account the valency of the interaction.

By “isolated” it is intended that the molecule, such as an antigen binding protein or nucleic acid, is removed from the environment in which it may be found in nature. For example, the molecule may be purified away from substances with which it would normally exist in nature. For example, the mass of the molecule in a sample may be 95% of the total mass.

The term “expression vector” as used herein means an isolated nucleic acid which can be used to introduce a nucleic acid of interest into a cell, such as a eukaryotic cell or prokaryotic cell, or a cell free expression system where the nucleic acid sequence of interest is expressed as a peptide chain such as a protein. Such expression vectors may be, for example, cosmids, plasmids, viral sequences, transposons, and linear nucleic acids comprising a nucleic acid of interest. Once the expression vector is introduced into a cell or cell free expression system (e.g., reticulocyte lysate) the protein encoded by the nucleic acid of interest is produced by the transcription/translation machinery. Expression vectors within the scope of the disclosure may provide necessary elements for eukaryotic or prokaryotic expression and include viral promoter driven vectors, such as CMV promoter driven vectors, e.g., pcDNA3.1, pCEP4, and their derivatives, Baculovirus expression vectors, Drosophila expression vectors, and expression vectors that are driven by mammalian gene promoters, such as human Ig gene promoters. Other examples include prokaryotic expression vectors, such as T7 promoter driven vectors, e.g., pET41, lactose promoter driven vectors and arabinose gene promoter driven vectors. Those of ordinary skill in the art will recognize many other suitable expression vectors and expression systems.

The term “recombinant host cell” as used herein means a cell that comprises a nucleic acid sequence of interest that was isolated prior to its introduction into the cell. For example, the nucleic acid sequence of interest may be in an expression vector while the cell may be prokaryotic or eukaryotic. Exemplary eukaryotic cells are mammalian cells, such as but not limited to, COS-1, COS-7, HEK293, BHK21, CHO, BSC-1, HepG2, 653, SP2/0, NS0, 293, HeLa, myeloma, lymphoma cells or any derivative thereof Most preferably, the eukaryotic cell is a HEK293, NSO, SP2/0, or CHO cell. E. coli is an exemplary prokaryotic cell. A recombinant cell according to the disclosure may be generated by transfection, cell fusion, immortalization, or other procedures well known in the art. A nucleic acid sequence of interest, such as an expression vector, transfected into a cell may be extrachromasomal or stably integrated into the chromosome of the cell.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl Acad Sci USA, 86:10029-10032 (1989), Hodgson, et al., Bio/Technology, 9:421 (1991)). A suitable human acceptor antibody may be one selected from a conventional database, e.g., the KABAT™ database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies—see, for example, EP-A-0239400 and EP-A-054951.

The term “fully human antibody” includes antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). Fully human antibodies comprise amino acid sequences encoded only by polynucleotides that are ultimately of human origin or amino acid sequences that are identical to such sequences. As meant herein, antibodies encoded by human immunoglobulin-encoding DNA inserted into a mouse genome produced in a transgenic mouse are fully human antibodies since they are encoded by DNA that is ultimately of human origin. In this situation, human immunoglobulin-encoding DNA can be rearranged (to encode an antibody) within the mouse, and somatic mutations may also occur. Antibodies encoded by originally human DNA that has undergone such changes in a mouse are fully human antibodies as meant herein. The use of such transgenic mice makes it possible to select fully human antibodies against a human antigen. As is understood in the art, fully human antibodies can be made using phage display technology wherein a human DNA library is inserted in phage for generation of antibodies comprising human germline DNA sequence.

The term “donor antibody” refers to an antibody that contributes the amino acid sequences of its variable regions, CDRs, or other functional fragments or analogs thereof to a first immunoglobulin partner. The donor, therefore, provides the altered immunoglobulin coding region and resulting expressed altered antibody with the antigenic specificity and neutralising activity characteristic of the donor antibody.

The term “acceptor antibody” refers to an antibody that is heterologous to the donor antibody, which contributes all (or any portion) of the amino acid sequences encoding its heavy and/or light chain framework regions and/or its heavy and/or light chain constant regions to the first immunoglobulin partner. A human antibody may be the acceptor antibody.

The terms “V_{H}” and “V_{L}” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

“Percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off.

“Percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDR(s). Alternatively, the % identity may exclude the CDR(s), for example the CDR(s) is 100% identical to the subject sequence and the % identity variation is in the remaining portion of the query sequence, so that the CDR sequence is fixed/intact.

In one aspect, methods of treating cancer in a patient in need thereof, comprising administering to the patient an effective amount of an agent directed to human ICOS and an effective amount of an agent directed to human OX40 sequentially are provided. In one embodiment, administration of the agent directed to human ICOS is prior to administration of the agent directed to human OX40. In another embodiment, administration of the agent directed to human OX40 is prior to administration of the agent directed to human ICOS. In one embodiment, the agent directed to human ICOS is an anti-ICOS antibody or antigen binding portion thereof. In one embodiment, the agent directed to human OX40 is an anti-OX40 antibody or antigen binding portion thereof.

In one aspect, an anti-ICOS antibody or antigen binding fragment thereof and an anti-OX40 antibody or antigen binding fragment thereof for sequential use in treating cancer in a human in need thereof are provided. In one embodiment, the anti-ICOS antibody or antigen binding fragment thereof is administered prior to administration of the anti-OX40 antibody or antigen binding fragment thereof. In another embodiment, the anti-OX40 antibody or antigen binding fragment thereof is administered prior to administration of the anti-ICOS antibody or antigen binding fragment thereof.

In another aspect, use of an anti-ICOS antibody or antigen binding portion thereof and an anti-OX40 antibody or antigen binding portion thereof in the manufacture of a medicament for the treatment of cancer is provided, wherein the anti-ICOS antibody or antigen binding portion thereof and an anti-OX40 antibody or antigen binding portion thereof are sequentially administered, and wherein administration of the anti-ICOS antibody or antigen binding portion thereof is followed by administration of the anti-OX40 antibody or antigen binding portion thereof.

The present invention also provides polynucleotides encoding anti-ICOS antibodies, anti-OX40 antibodies, or antigen binding portion of any one of said antibodies, of the present invention. In one embodiment, host cells are provided comprising polynucleotides encoding anti-ICOS antibodies, anti-OX40 antibodies, or antigen binding portions of any one of said antibodies, of the present invention. The present invention also provides methods of making an anti-ICOS antibody, anti-OX40 antibody, or an antigen binding portion of said antibody, comprising the steps of a) culturing host cell comprising a polynucleotide encoding an anti-ICOS antibody, anti-OX40 antibody, or an antigen binding portion of said antibody of the present invention under suitable conditions to express said anti-ICOS antibody, anti-OX40 antibody, or antigen binding portion of said antibody; and b) isolating said anti-ICOS, anti-OX40, or antigen binding portion of said antibody.

In another aspect, a polynucleotide encoding an anti-ICOS antibody or antigen binding portion thereof is provided, wherein the anti-ICOS antibody or antigen binding portion thereof is sequentially administered to a cancer patient with an anti-OX40 antibody or antigen binding portion thereof, and wherein administration of the anti-ICOS antibody or antigen binding portion thereof is followed by administration of the anti-OX40 antibody or antigen binding portion thereof.

In yet another aspect, a polynucleotide encoding an anti-OX40 antibody or antigen binding portion thereof is provided, wherein the anti-OX40 antibody or antigen binding portion thereof is sequentially administered to a cancer patient with an anti-ICOS antibody or antigen binding portion thereof, and wherein administration of the anti-ICOS antibody or antigen binding portion thereof is followed by administration of the anti-OX40 antibody or antigen binding portion thereof.

In another aspect, a vector comprising the polynucleotide of any one of the aspects herein is provided. In another aspect, a host cell comprising the vector of any one of the aspects herein is provided.

In yet another aspect, a method of making an anti-ICOS antibody or antigen binding portion thereof is provided, the method comprising a) culturing a host cell comprising the polynucleotide of any one of the aspects herein under suitable conditions to express the anti-ICOS antibody or antigen binding portion thereof; and b) isolating said anti-ICOS antibody or antigen binding portion thereof.

In another aspect, a method of making an anti-OX40 antibody or antigen binding portion thereof is provided, the method comprising a) culturing a host cell comprising the polynucleotide of any one of the aspects herein under suitable conditions to express the anti-OX40 antibody or antigen binding portion thereof; and b) isolating said anti-OX40 antibody or antigen binding portion thereof.

In one embodiment of any one of the aspects herein, the anti-ICOS antibody is an ICOS agonist. In one embodiment, the anti-ICOS antibody comprises a V_{H} domain comprising an amino acid sequence at least 90% identical to the amino acid sequence set forth in SEQ ID NO:46; and a V_{L} domain comprising an amino acid sequence at least 90% identical to the amino acid sequence as set forth in SEQ ID NO:47. In another embodiment, the the anti-ICOS antibody comprises a V_{H} domain comprising the amino acid sequence set forth in SEQ ID NO:46 and a VL domain comprising the amino acid sequence as set forth in SEQ ID NO:47. In one embodiment, the anti-ICOS antibody comprises one or more of: CDRH1 as set forth in SEQ ID NO:40; CDRH2 as set forth in SEQ ID NO:41; CDRH3 as set forth in SEQ ID NO:42; CDRL1 as set forth in SEQ ID NO:43; CDRL2 as set forth in SEQ ID NO:44 and/or CDRL3 as set forth in SEQ ID NO:45 or a direct equivalent of each CDR wherein a direct equivalent has no more than two amino acid substitutions in said CDR.

In one embodiment of any one of the aspects herein, the anti-OX40 antibody is an OX40 agonist. In one embodiment, the anti-OX40 antibody comprises a V_{H} domain comprising an amino acid sequence at least 90% identical to the amino acid sequence set forth in SEQ ID NO:5; and a V_{L} domain comprising an amino acid sequence at least 90% identical to the amino acid sequence as set forth in SEQ ID NO:11. In another embodiment, the the anti-OX40 antibody comprises a V_{H} domain comprising the amino acid sequence set forth in SEQ ID NO:5 and a V_{L} domain comprising the amino acid sequence as set forth in SEQ ID NO:11. In one embodiment, the anti-OX40 antibody comprises one or more of: CDRH1 as set forth in SEQ ID NO:1; CDRH2 as set forth in SEQ ID NO:2; CDRH3 as set forth in SEQ ID NO:3; CDRL1 as set forth in SEQ ID NO:7; CDRL2 as set forth in SEQ ID NO:8 and/or CDRL3 as set forth in SEQ ID NO:9 or a direct equivalent of each CDR wherein a direct equivalent has no more than two amino acid substitutions in said CDR.

In one embodiment of any one of the aspects herein, the agent directed to human ICOS is administered for 2, 3, 4, 5, 6, 7, 8, 9, 10, 11, 12, 13, 14, 15, 16, 17, 18, 19, 20, 21, 22, 23, 24, 25, 26, 27, 28, 29, or 30 consecutive days. In one embodiment of any one of the aspects herein, the agent directed to human OX40 is administered for 2, 3, 4, 5, 6, 7, 8, 9, 10, 11, 12, 13, 14, 15, 16, 17, 18, 19, 20, 21, 22, 23, 24, 25, 26, 27, 28, 29, or 30 consecutive days.

In one aspect, the cancer is selected from the group consisting of colorectal cancer (CRC), gastric, esophageal, cervical, bladder, breast, head and neck, ovarian, melanoma, renal cell carcinoma (RCC), EC squamous cell, non-small cell lung carcinoma, mesothelioma, pancreatic, and prostate cancer.

In another aspect the cancer is selected from head and neck cancer, breast cancer, lung cancer, colon cancer, ovarian cancer, prostate cancer, gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma. Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), and testicular cancer.

Some embodiments of the present invention further comprise administering at least one neo-plastic agent and/or at least one immunostimulatory agent to said human.

In one aspect the human has a solid tumor. In one aspect the tumor is selected from head and neck cancer, gastric cancer, melanoma, renal cell carcinoma (RCC), esophageal cancer, non-small cell lung carcinoma, prostate cancer, colorectal cancer, ovarian cancer and pancreatic cancer. In another aspect the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

By the term “treating” and grammatical variations thereof as used herein, is meant therapeutic therapy. In reference to a particular condition, treating means: (1) to ameliorate the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy using the methods and/or compositions of the invention is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the terms “cancer,” “neoplasm,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (NTS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent Bcell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphomas (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

As used herein the term “Compound A²” means an agent directed to human ICOS. In some embodiments, Compound A² is an antibody to human ICOS or the antigen binding portion thereof. In some embodiments, Compound A² is an ICOS agonist. Suitably Compound A² means a humanized monoclonal antibody having a heavy chain variable region as set forth in SEQ ID NO:46 and a light chain variable region as set forth in SEQ ID NO:47.

As used herein the term “Compound B²” means an agent directed to human OX40. In some embodiments, Compound B² is an OX40 agonist. In some embodiments, Compound B² is an antibody to human OX40 or the antigen binding portion thereof. Suitably, Compound B² means a humanized monoclonal antibody having a heavy chain variable region as set forth in SEQ ID NO:5 and a light chain variable region as set forth in SEQ ID NO:11.

Suitably, the combinations of this invention are administered within a “specified period”.

The term “specified period” and grammatical variations thereof, as used herein, means the interval of time between the administration of one of Compound A² and Compound B² and the other of Compound A² and Compound B².

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

The term “duration of time” and grammatical variations thereof, as used herein means that both compounds of the invention are administered for an indicated number of consecutive days. Unless otherwise defined, the number of consecutive days does not have to commence with the start of treatment or terminate with the end of treatment, it is only required that the number of consecutive days occur at some point during the course of treatment.

### Regarding “Specified Period” Administration:

Suitably, both compounds will be administered within a specified period for at least one day—in this case, the duration of time will be at least one day; suitably, during the course to treatment, both compounds will be administered within a specified period for at least 3 consecutive days—in this case, the duration of time will be at least 3 days; suitably, during the course to treatment, both compounds will be administered within a specified period for at least 5 consecutive days—in this case, the duration of time will be at least 5 days; suitably, during the course to treatment, both compounds will be administered within a specified period for at least 7 consecutive days—in this case, the duration of time will be at least 7 days; suitably, during the course to treatment, both compounds will be administered within a specified period for at least 14 consecutive days—in this case, the duration of time will be at least 14 days; suitably, during the course to treatment, both compounds will be administered within a specified period for at least 30 consecutive days—in this case, the duration of time will be at least 30 days.

Suitably, if the compounds are not administered during a “specified period”, they are administered sequentially. By the term “sequential administration”, and grammatical derivates thereof, as used herein is meant that one of Compound A² and Compound B² is administered for two or more consecutive days and the other of Compound A² and Compound B² is subsequently administered for two or more consecutive days. During the period of consecutive days in which Compound A² is administered, at least 1 dose, at least 2 doses, at least 3 doses, at least 4 doses, at least 5 doses, at least 6 doses, at least 7 doses, at least 8 doses, at least 9 doses, or at least 10 doses of Compound A² is administered. During the period of consecutive days in which Compound B² is administered, at least 1 dose, at least 2 doses, at least 3 doses, at least 4 doses, at least 5 doses, at least 6 doses, at least 7 doses, at least 8 doses, at least 9 doses, or at least 10 doses Compound B² is administered. During the period of consecutive days in which Compound A² is administered, Compound A² can be administered at least three times a day, at least twice a day, at least once a day, or less than once a day, e.g., once every 2 days, once every 3 days, once every week, once every 2 weeks, once every 3 weeks, or once every 4 weeks. During the period of consecutive days in which Compound B² is admininstered, Compound B² can be administered at least three times a day, at least twice a day, at least once a day, or less than once a day, e.g., once every 2 days, once every 3 days, once every week, once every 2 weeks, once every 3 weeks, or once every 4 weeks.

Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B² is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

Sequential administration can also include one of Compound A² and Compound B² is administered for two or more consecutive days and then both of Compound A² and Compound B² is subsequently administered for two or more consecutive days. Sequential administration can include both of Compound A² and Compound B² being administered for two or more consecutive days and then one of Compound A² and Compound B² being subsequently administered for two or more consecutive days

### Regarding Sequential Administration:

Suitably, one of Compound A² and Compound B² is administered for from 1 to 30 consecutive days, followed by an optional drug holiday, followed by administration of the other of Compound A² and Compound B² for from 1 to 30 consecutive days. Suitably, one of Compound A² and Compound B² is administered for from 1 to 21 consecutive days, followed by an optional drug holiday, followed by administration of the other of Compound A² and Compound B² for from 1 to 21 consecutive days. Suitably, one of Compound A² and Compound B² is administered for from 1 to 14 consecutive days, followed by a drug holiday of from 1 to 14 days, followed by administration of the other of Compound A² and Compound B² for from 1 to 14 consecutive days. Suitably, one of Compound A² and Compound B² is administered for from 1 to 7 consecutive days, followed by a drug holiday of from 1 to 10 days, followed by administration of the other of Compound A² and Compound B² for from 1 to 7 consecutive days.

Suitably, Compound B² will be administered first in the sequence, followed by an optional drug holiday, followed by administration of Compound A². Suitably, Compound B² is administered for from 3 to 21 consecutive days, followed by an optional drug holiday, followed by administration of Compound A² for from 3 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A² for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably, Compound B² is administered for 3 consecutive days, followed by a drug holiday of from 3 to 10 days, followed by administration of Compound A² for 3 consecutive days.

It is understood that a “specified period” administration and a “sequential” administration can be followed by repeat dosing or can be followed by an alternate dosing protocol, and a drug holiday may precede the repeat dosing or alternate dosing protocol.

The methods of the present invention may also be employed with other therapeutic methods of cancer treatment.

Compound A² and Compound B² may be administered by any appropriate route. Suitable routes include oral, rectal, nasal, topical (including buccal and sublingual), intratumorally, vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation.

In one embodiment, one or more components of a combination of the invention are administered intravenously. In one embodiment, one or more components of a combination of the invention are administered orally. In another embodiment, one or more components of a combination of the invention are administered intratumorally. In another embodiment, one or more components of a combination of the invention are administered systemically, e.g., intravenously, and one or more other components of a combination of the invention are administered intratumorally. In any of the embodiments, e.g., in this paragraph, the components of the invention are administered as one or more pharmaceutical compositions.

In one aspect methods are provided for the treatment of cancer, comprising administering to a human in need thereof a therapeutically effective amount of (i) an anti-ICOS antibody or the antigen binding portion thereof, in addition to one of more diluents, vehicles, excipients and/or inactive ingredients, and (ii) an anti-OX40 antibody or the antigen binding portion thereof or the antigen binding portion thereof, in addition to one of more diluents, vehicles, excipients and/or inactive ingredients. In one embodiment sequential administration of an anti-ICOS antibody or the antigen binding portion thereof and an anti-OX40 antibody or antigen binding portion thereof provides a synergistic effect compared to administration of either agent as monotherapy or concurrently.

In one embodiment, the anti-ICOS antibody or antigen binding portion thereof comprises a V_{H} domain comprising an amino acid sequence at least 90% identical to the amino acid sequence set forth in SEQ ID NO:7; and a V_{L} domain comprising an amino acid sequence at least 90% identical to the amino acid sequence as set forth in SEQ ID NO:8.

In one embodiment, methods of treating cancer are provided wherein the anti-ICOS antibody or antigen binding portion thereof is administered at a time interval selected from once every week, once every two weeks, once every three weeks, and once every four weeks. In another embodiment, the anti-OX40 antibody or antigen binding portion thereof is administered at a time interval selected from once every week, once every two weeks, once every three weeks, and once every four weeks. As is understood in the art the start of administration of either agent can be separated by an interstitial period. The interstitial period may be 12 hours, one to six days, one week, two weeks, three weeks, four weeks, five weeks, or six weeks. By way of example, an anti-ICOS antibody could be administered on Day 1 of treatment with an interstitial period of two weeks before the start of anti-OX40 antibody therapy which would start on Day 14. In one aspect, treatment with said anti-ICOS antibody could continue with administration of a single IV infusion at a time interval of, for example, every one, two, three or four weeks. Similarly, treatment with said anti-OX40 antibody could continue with administration of a single IV infusion at a time interval of, for example, every one, two, three or four weeks.

In one embodiment, the anti-ICOS antibody or antigen binding portion thereof is administered as an IV infusion. In one embodiment, the anti-OX40 antibody or antigen binding portion thereof is administered as an IV infusion. In one aspect, the anti-ICOS antibody or antigen binding portion thereof is administered prior to the anti-OX40 antibody or the antigen binding portion thereof. In one embodiment, administration of the anti-OX40 antibody or antigen binding portion thereof is initiated at a time point selected from 1 week, 2 weeks, 3 weeks, and 4 weeks after the start of the administration of said anti-ICOS antibody or antigen binding portion thereof. In one aspect, the anti-OX40 antibody or antigen binding portion thereof is administered prior to the anti-ICOS antibody or the antigen binding portion thereof. In one embodiment, the interstitial period between the start of the anti-OX40 antibody or anti-OX40 therapy and the start of the anti-ICOS antibody therapy is selected from 1 day, 1 week, 2 weeks, 3 weeks, 4 weeks, 5 weeks, and 6 weeks.

In one embodiment, the anti-ICOS antibody or antigen binding portion thereof and said anti-OX40 antibody or antigen binding portion thereof are administered to said human until said human shows disease progression or unacceptable toxicity. In one embodiment, methods are provided for the treatment of cancer further comprising administering at least one anti neoplastic agent and/or at least one immuno- modulatory agent to said human.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita, T. S. Lawrence, and S .A. Rosenberg (editors), 10^{th} edition (Dec. 5, 2014), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule or anti-mitotic agents such as diterpenoids and vinca alkaloids; platinum coordination complexes; alkylating agents such as nitrogen mustards, oxazaphosphorines, alkylsulfonates, nitrosoureas, and triazenes; antibiotic agents such as actinomycins, anthracyclins, and bleomycins; topoisomerase I inhibitors such as camptothecins; topoisomerase II inhibitors such as epipodophyllotoxins; antimetabolites such as purine and pyrimidine analogues and anti-folate compounds; hormones and hormonal analogues; signal transduction pathway inhibitors; non-receptor tyrosine kinase angiogenesis inhibitors; immunotherapeutic agents; proapoptotic agents; cell cycle signaling inhibitors; proteasome inhibitors; heat shock protein inhibitors; inhibitors of cancer metabolism; and cancer gene therapy agents such as genetically modified T cells.

Examples of a further active ingredient or ingredients for use in combination or co-administered with the present methods or combinations are anti-neoplastic agents. Examples of anti-neoplastic agents include, but are not limited to, chemotherapeutic agents; immuno-modulatory agents; immuno-modulators; and immunostimulatory adjuvants.

### EXAMPLES

The following examples illustrate various non-limiting aspects of this invention.

### Example 1: Anti-ICOS Antibody Treatment Increases OX40 Expression on T Cells; Anti-OX40 Antibody Treatment Increases ICOS Expression on T Cells

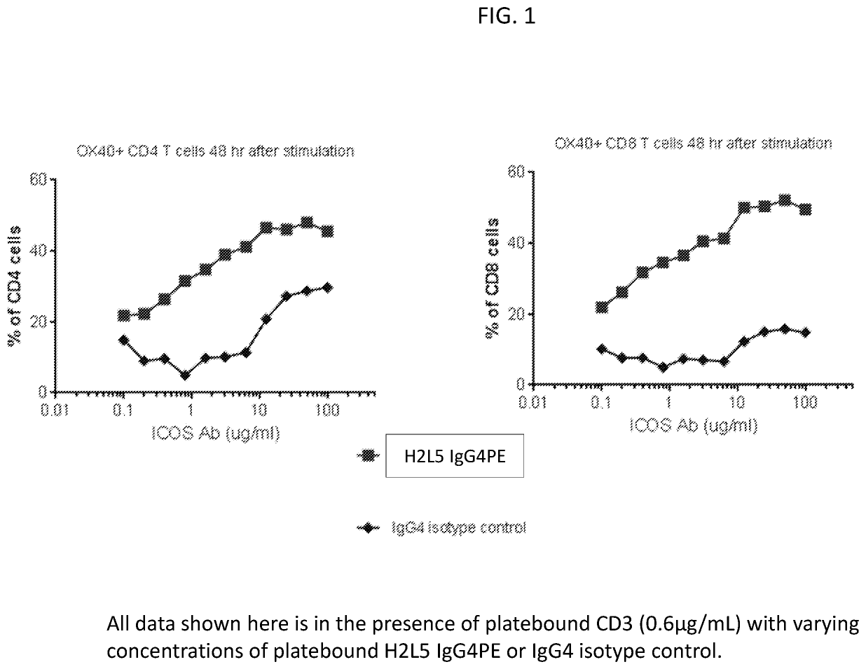

As shown in FIG. 1, anti-ICOS antibody (H2L5 IgG4PE) concentration dependent increase in OX40+ CD4 and CD8 T cells was observed. Data shown in FIG. 1 was obtained in the presence of platebound anti-CD3 (0.6 μg/mL) with varying concentrations of platebound H2L5 IgG4PE or IgG4 isotype control.

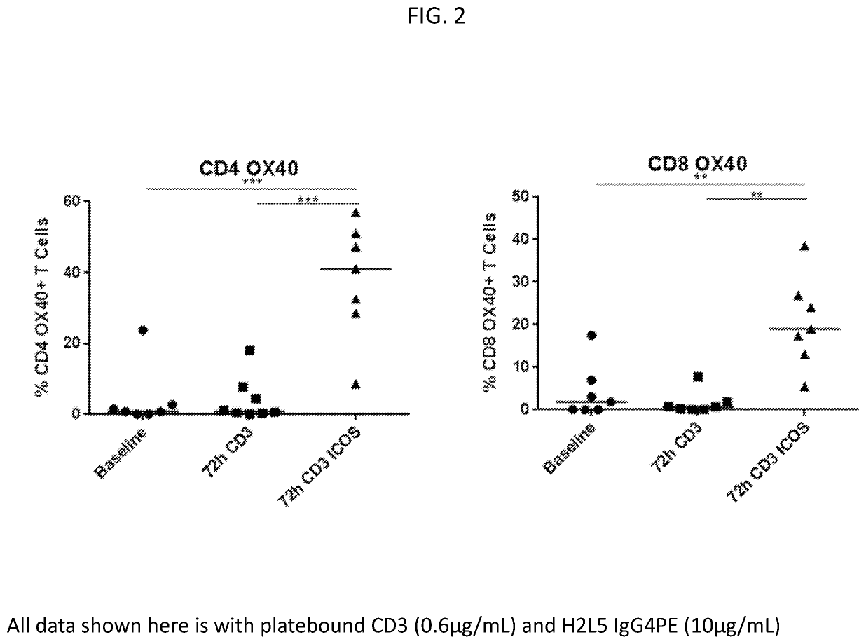

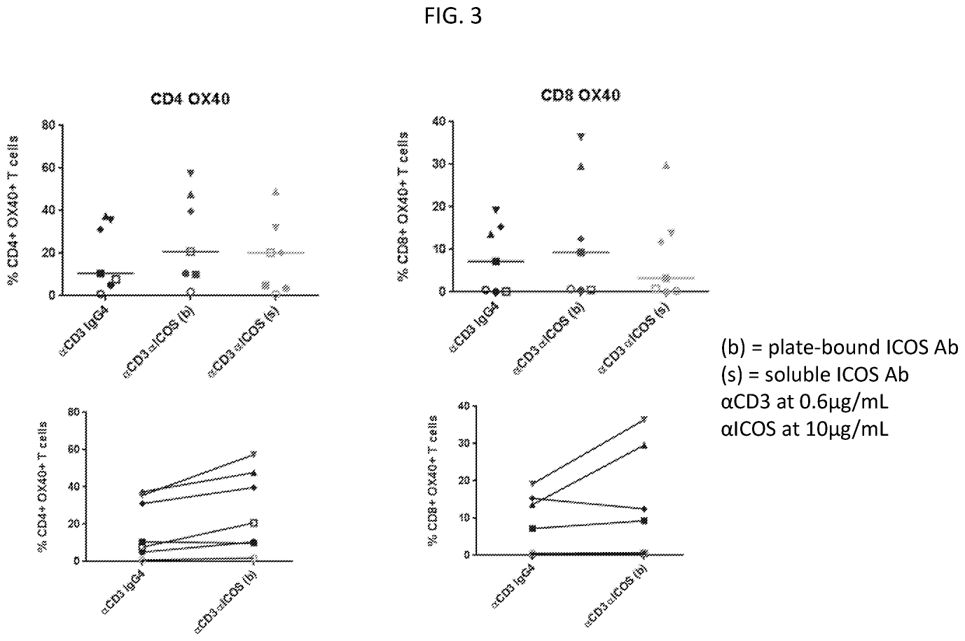

Anti-ICOS antibody (H2L5 IgG4PE) treatment increased OX40+ CD4 and CD8 T cells in in vitro assays with cancer patient PBMC (FIG. 2). Data shown in FIG. 2 was with platebound anti-CD3 (0.6 μg/mL) and H2L5 IgG4PE (10 μg/mL). Anti-ICOS antibody (H2L5 IgG4PE) treatment increased OX40+ CD4 and CD8 T cells in expanded TIL (tumor infiltrating lymphocyte) cultures (anti-CD3 at 0.6 μg/mL, and H2L5 IgG4PE at 10 μg/mL) (FIG. 3).

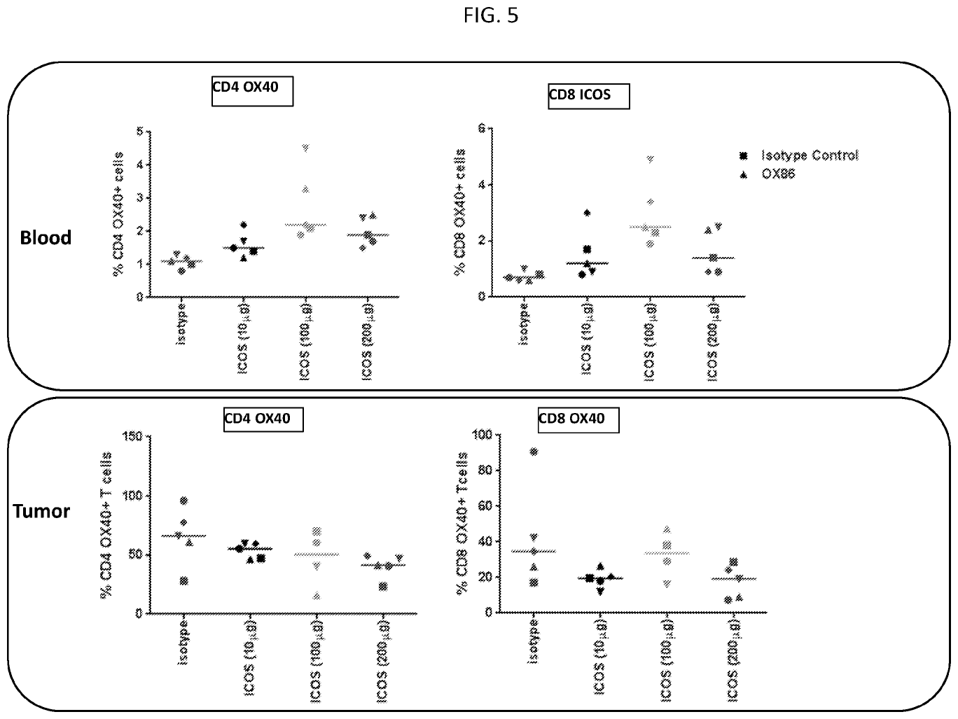

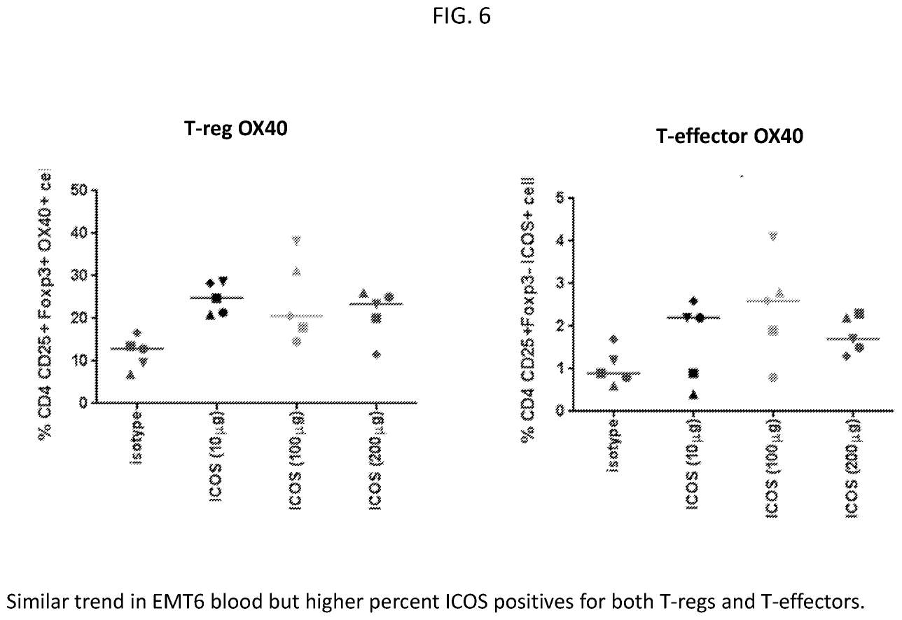

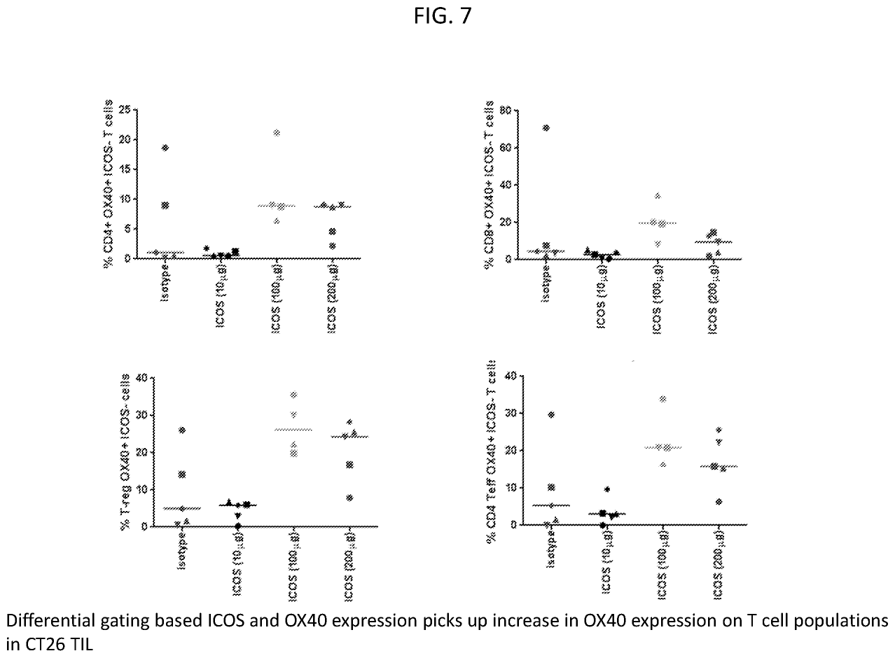

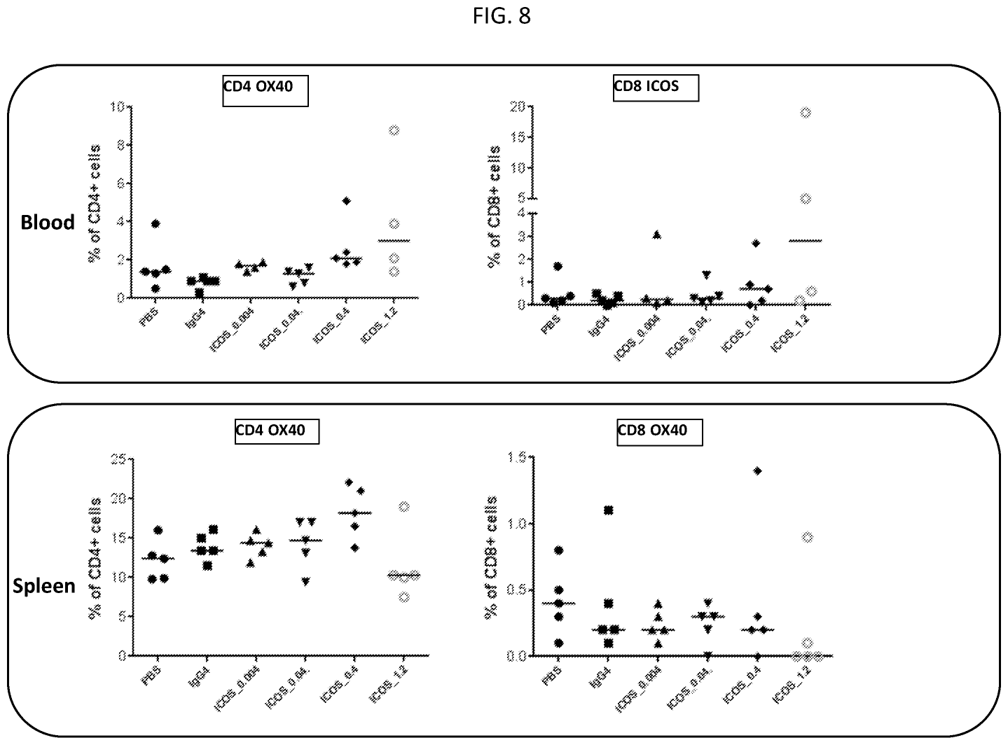

In CT26 tumor bearing mice, anti-ICOS antibody treatment increased OX40+ T cells in blood (FIG. 5). Anti-ICOS antibody treatment increased OX40+ T-reg and CD4 T-effectors in blood from CT26 tumor bearing mice (FIG. 6). A similar trend in EMT6 blood was observed, but with a higher percent ICOS positives for both T-regs and T-effectors. Anti-ICOS antibody treatment increased OX40+ ICOS− T-cells in tumors from CT26 tumor bearing mice (FIG. 7). Differential gating based on ICOS and OX40 expression picked up increase in OX40 expression on T cell populations in CT26 TIL. Changes in OX40+ T cells in blood and spleens from ICOS treated A2058 melanoma tumors in huPBMC (human peripheral blood mononuclear cell) model were observed (FIG. 8).

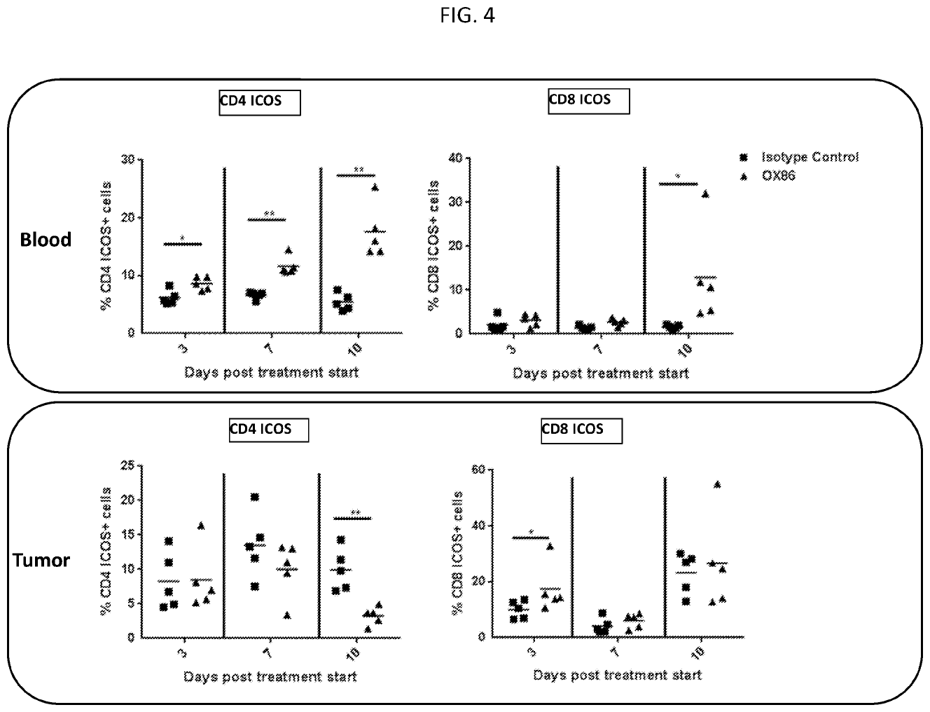

Anti-OX40 antibody treatment increased ICOS+ CD4 and CD8 T cells in blood while decreasing ICOS+ CD4 in tumors from CT26 tumor bearing mice (FIG. 4).

### Example 2: Anti-ICOS Antibody/Anti-OX40 Antibody Concurrent and Phased Dosing Study

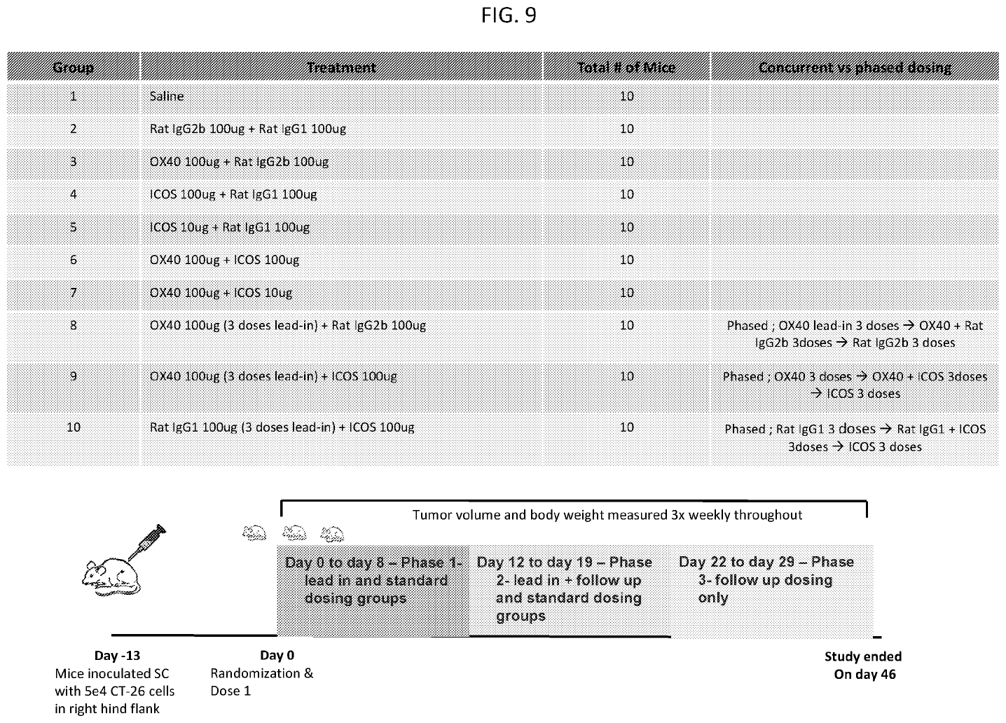

Efficacy of anti-ICOS antibody (17G9 clone) and anti-OX40 antibody (OX86 clone) was studied in a CT26 syngeneic model. FIG. 9 shows the study design of an anti-ICOS antibody (17G9 clone)/anti-OX40 antibody (OX86 clone) concurrent and phased dosing study conducted. 5.0×10⁴ cells/mouse of CT26 mouse colon carcinoma tumor cells were inoculated subcutaneously into the right hind flank. Dosing started on randomization day. Concurrent and phased dosing were carried out as shown the table and schematic in FIG. 9.

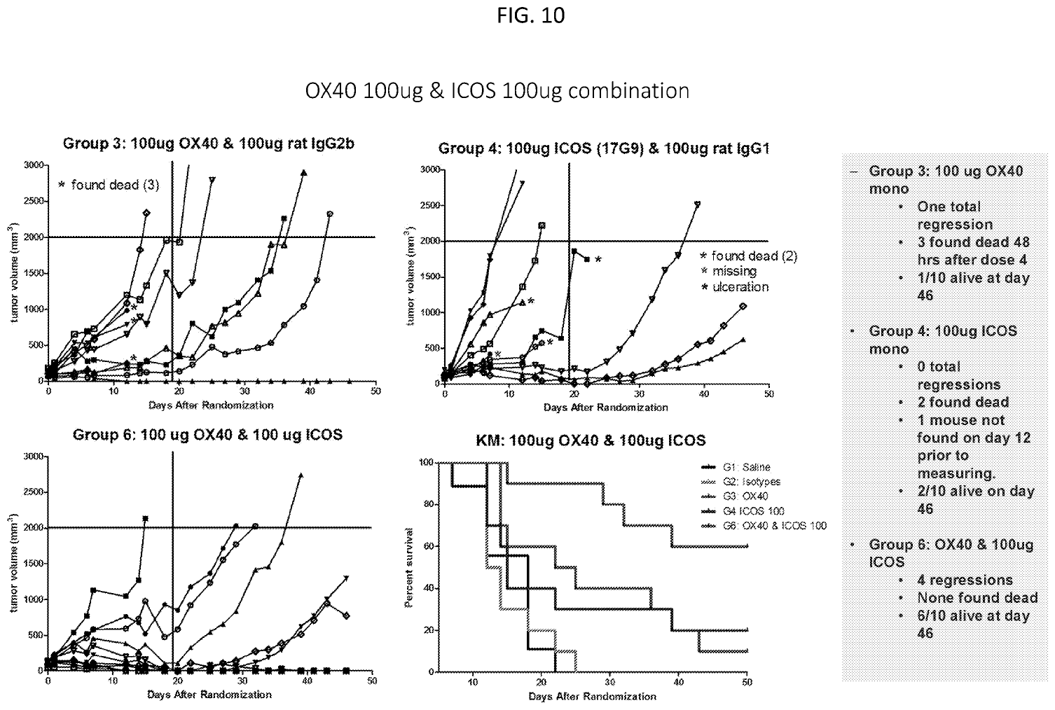

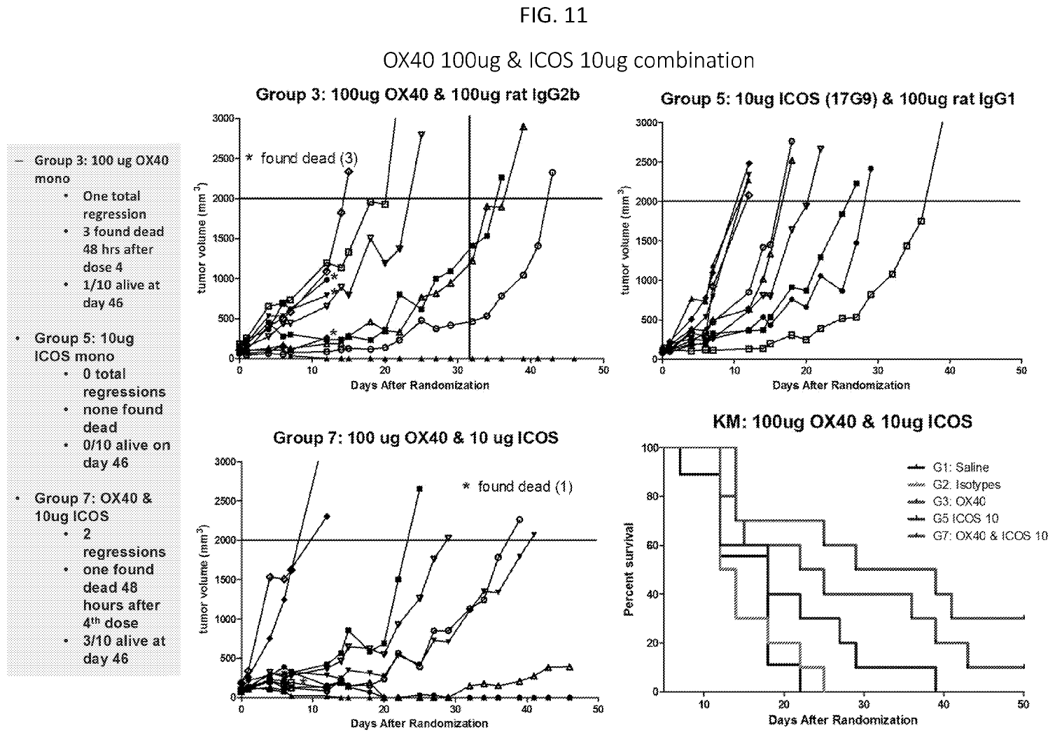

Tumor volume and survival in groups treated with concurrent dosing of 100 μg or 10 μg anti-ICOS antibody and 100 μg anti-OX40 antibody combination and treated with anti-ICOS or anti-OX40 monotherapy with appropriate isotype controls are shown in FIGS. 10-11. Group 3 received 100 μg anti-OX40 monotherapy. One total regression was observed; 3 mice were found dead 48 hours after dose 4, and 1/10 were alive at day 46. Group 4 received 100 μg anti-ICOS monotherapy. There were 0 total regression, 2 found dead, 1 mouse not found on day 12 prior to measuring, and 2/10 alive on day 46. Group 5 received 10 μg anti-ICOS monotherapy. There were 0 total regressions, none found dead, 0/10 alive on day 46. Group 6 received 100 μg anti-OX40 and 100 μg anti-ICOS combination. There were 4 regressions observed, none found dead, and 6/10 alive at day 46. Group 7 received 10 μg anti-ICOS and 100 μg anti-OX40 combination. There were 2 regressions, one found dead 48 hours after the 4^{th} dose, and 3/10 alive at day 46. A synergistic effect on survival in the anti-ICOS antibody and anti-OX40 antibody combination was observed, as compared to each of the anti-OX40 and anti-ICOS monotherapy (FIG. 10).

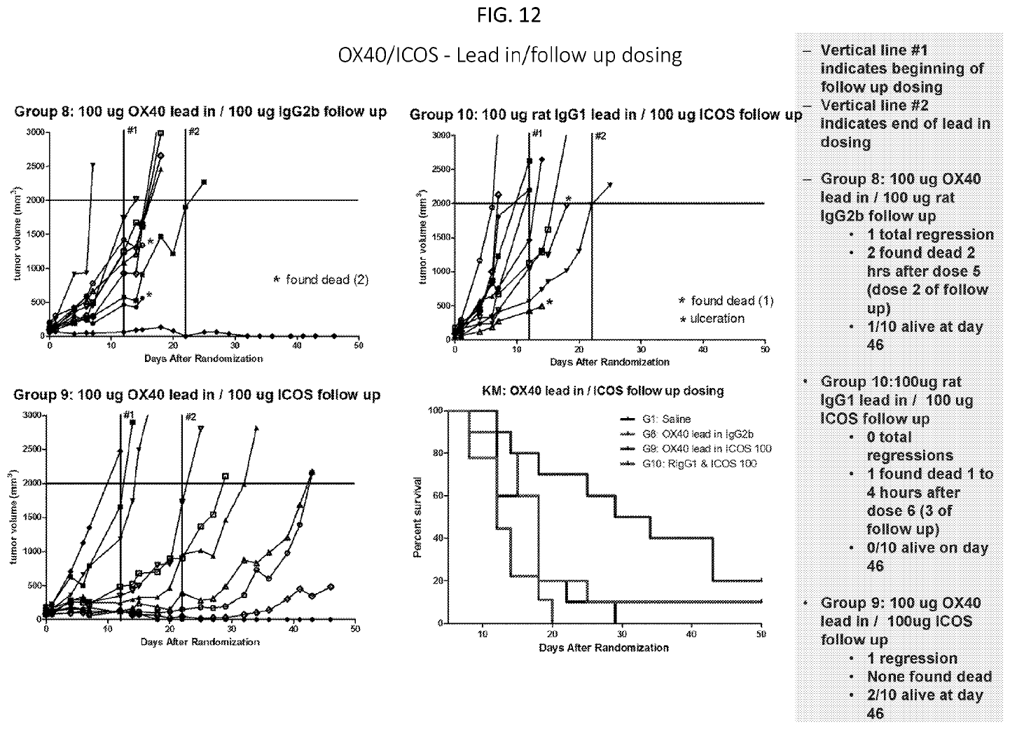

FIG. 12 shows tumor volume and survival of groups treated with phased dosing of anti-ICOS antibody and anti-OX40 antibody with 100 μg anti-OX40 lead in/100 μg anti-ICOS follow up (Group 9), and appropriate controls (Group 8: 100 μg anti-OX40 lead in/100 μg IgG2b follow up; Group 10: 100 μg rat IgG1 lead in/100 μg anti-ICOS follow up). In the plots showing tumor volume in FIG. 12, vertical line #1 indicates the beginning of follow up dosing and vertical line #2 indicates the end of lead in dosing.

Group 8 received 100 μg anti-OX40 lead in and 100 μg rat IgG2b follow up. There was 1 total regression observed, 2 found dead 2 hours after dose 5 (dose 2 of follow up), and 1/10 alive at day 46. Group 10 received 100 μg rat IgG1 lead in and 100 μg anti-ICOS follow up. There were 0 total regressions observed, 1 found dead 1 to 4 hours after dose 6 (dose 3 of follow up), and 0/10 alive on day 46. Group 9 received 100 μg anti-OX40 lead in and 100 μg anti-ICOS follow up. There was 1 regression, none found dead, and 2/10 alive at day 46.

### Example 3: ICOS and OX40 Expression on T Cells

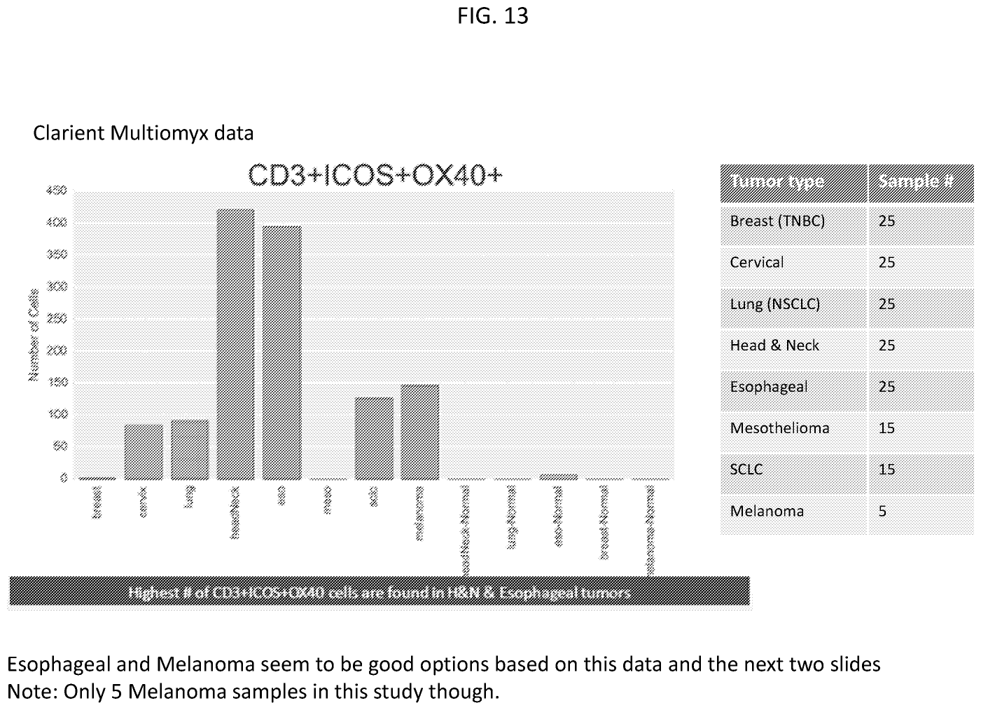

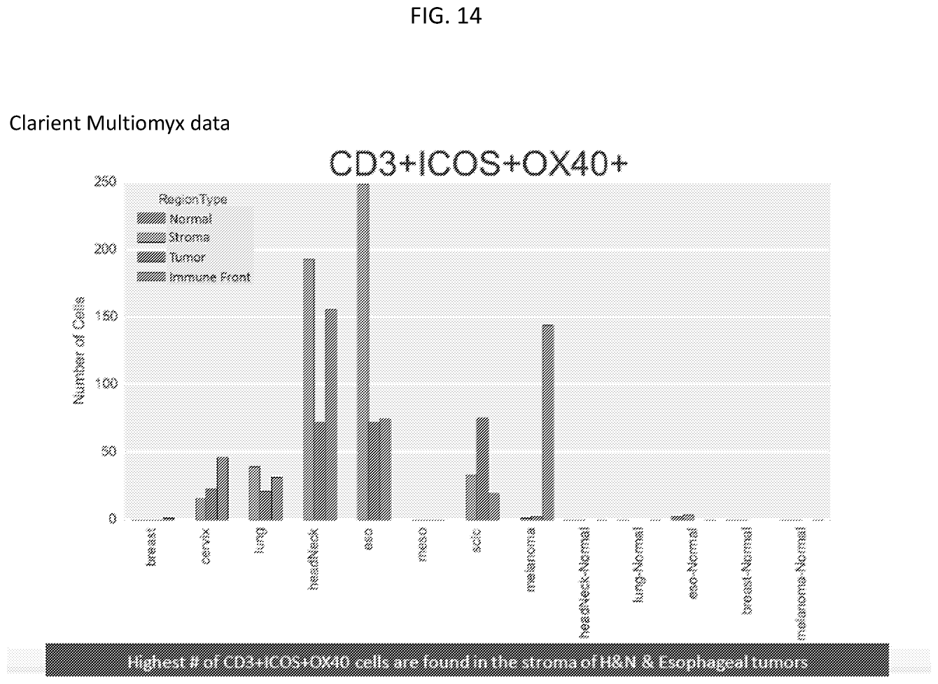

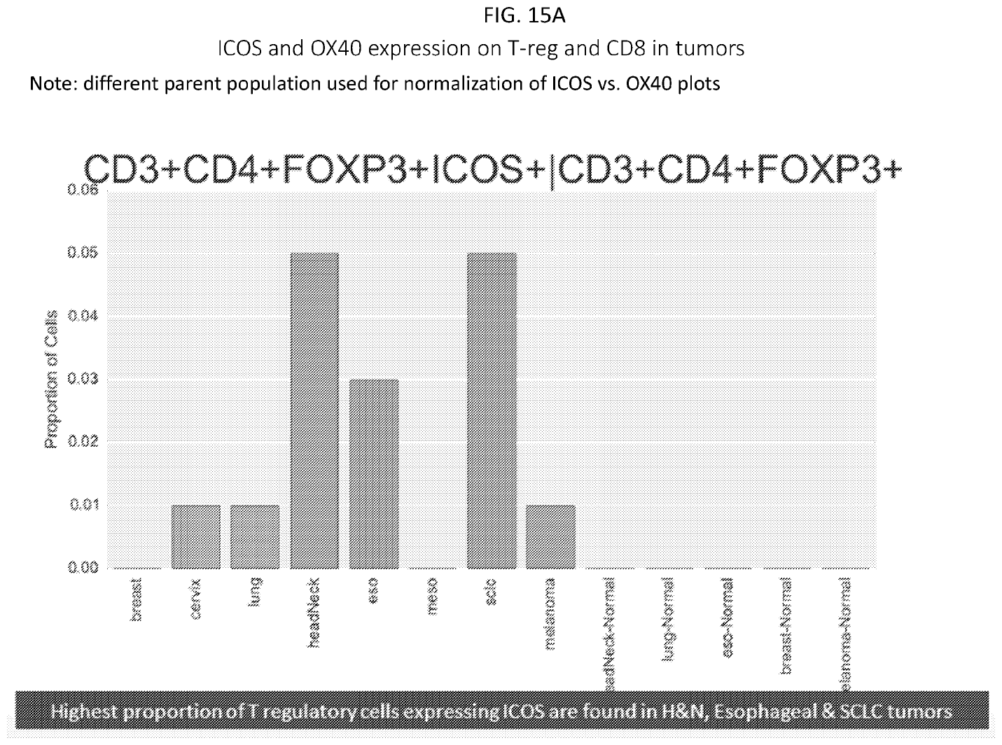

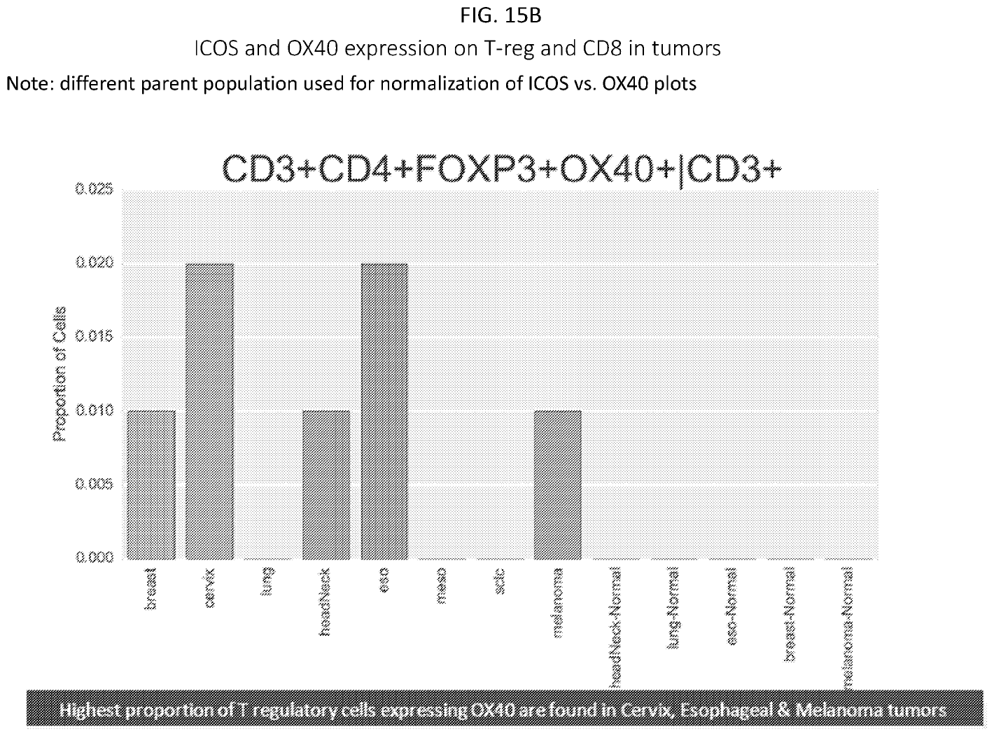

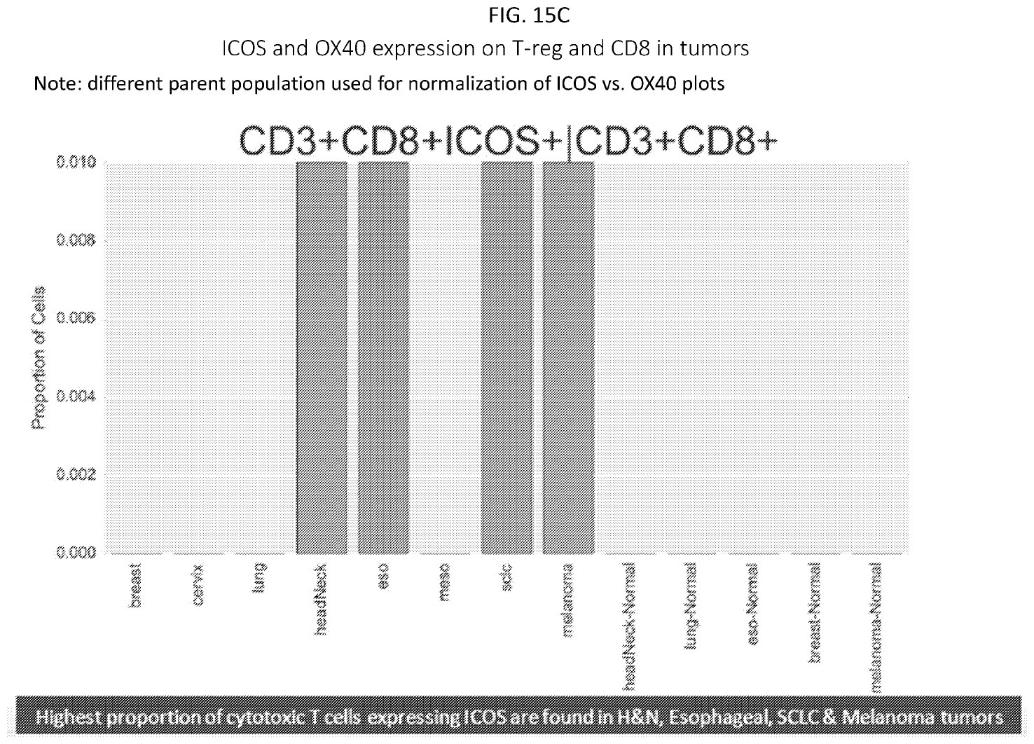

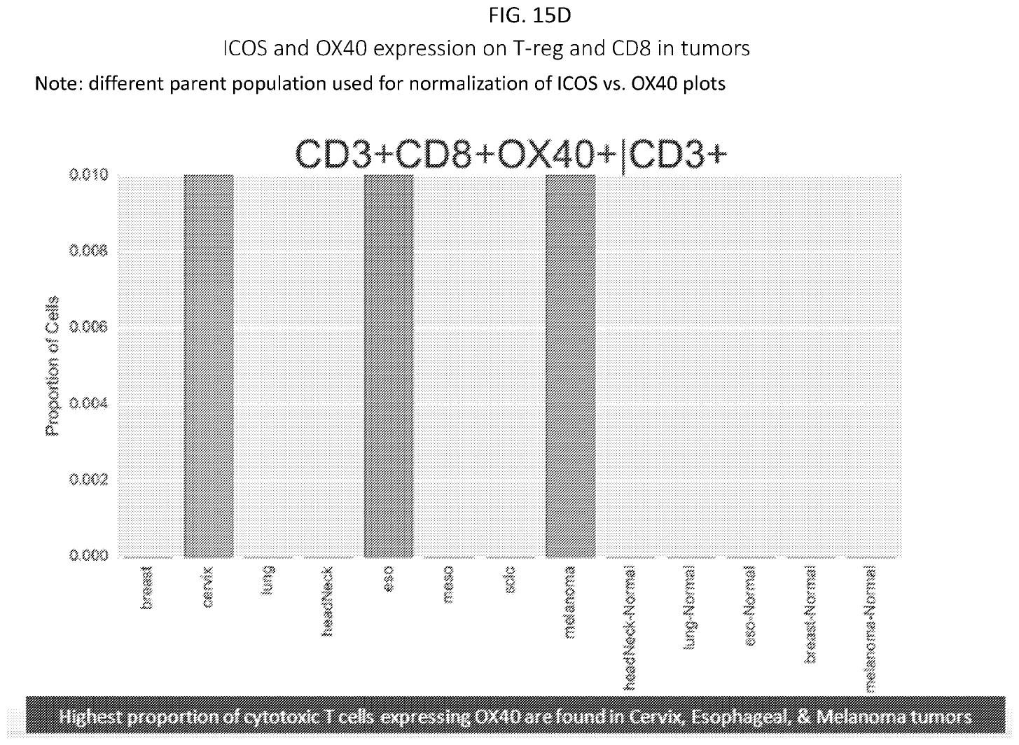

FIG. 13 shows tumors expressing ICOS and OX40 dual positive T cells. Esophageal and melanoma showed the highest numbers of ICOS and OX40 dual positive T cells; however, only 5 melanoma samples were used in the study. FIG. 14 shows data (Clarient Multiomyx) showing further separation of tumors based on regions in the TME (tumor microenvironment). In FIGS. 15A-15D, ICOS and OX40 expression on T-reg and CD8 in tumors is shown. Different parent populations were used for normalization of ICOS vs. OX40 plots. The highest proportion of T regulatory cells (T-reg cells) expressing ICOS were found in head and neck, esophageal, and SCLC (small cell lung cancer) tumors (FIG. 15A). The highest proportion of T regulatory cells expression OX40 were found in cervix, esophageal, and melanoma tumors (FIG. 15B). The highest proportion of cytotoxic T cells expressing ICOS were found in head and neck, esophageal, SCLC, and melanoma tumors (FIG. 15C). The highest proportion of cytotoxic T cells expressing OX40 were found in cervix, esophageal, and melanoma tumors (FIG. 15D).

1. A method of treating cancer in a patient in need thereof, the method comprising administering to the patient an effective amount of an agent directed to human ICOS and an effective amount of an agent directed to human OX40 sequentially.

2. The method of claim 1, wherein administration of the agent directed to human ICOS is prior to administration of the agent directed to human OX40.

3. The method of claim 1, wherein administration of the agent directed to human OX40 is prior to administration of the agent directed to human ICOS.

4. The method of any one of claims 1-3, wherein the agent directed to human ICOS is an anti-ICOS antibody or antigen binding portion thereof.

5. The method of any one of claims 1-4, wherein the agent directed to human ICOS or the anti-ICOS antibody or antigen binding portion thereof is an ICOS agonist.

6. The method of any one of claims 4-5, wherein the anti-ICOS antibody comprises a V_{H} domain comprising an amino acid sequence at least 90% identical to the amino acid sequence set forth in SEQ ID NO:46; and a V_{L} domain comprising an amino acid sequence at least 90% identical to the amino acid sequence as set forth in SEQ ID NO:47.

7. The method of any one of claims 4-6, wherein the anti-ICOS antibody comprises a V_{H} domain comprising the amino acid sequence set forth in SEQ ID NO:46 and a V_{L} domain comprising the amino acid sequence as set forth in SEQ ID NO:47.

8. The method of any one of claims 1-7, wherein the agent directed to human OX40 is an anti-OX40 antibody or antigen binding portion thereof.

9. The method of any one of claims 1-8, wherein the agent directed to human OX40 or anti-OX40 antibody or antigen binding portion thereof is an OX40 agonist.

10. The method of any one of claims -8-9, wherein the anti-OX40 antibody comprises a V_{H} domain comprising an amino acid sequence at least 90% identical to the amino acid sequence set forth in SEQ ID NO:5; and a V_{L} domain comprising an amino acid sequence at least 90% identical to the amino acid sequence as set forth in SEQ ID NO:11.

11. The method of any one of claims 8-10, wherein the anti-OX40 antibody comprises a V_{H} domain comprising the amino acid sequence set forth in SEQ ID NO:5 and a V_{L} domain comprising the amino acid sequence as set forth in SEQ ID NO:11.

12. The method of any one of claims 1-11, wherein the agent directed to human ICOS or the anti-ICOS antibody or antigen binding portion thereof is administered once every week, once every two weeks, once every three weeks, or once every four weeks.

13. The method of any one of claims 1-12, wherein the agent directed to human OX40 or the anti-OX40 antibody or antigen binding portion thereof is administered once every week, once every two weeks, once every three weeks, or once every four weeks.

14. The method of any one of claims 1-13, wherein the cancer is selected from the group consisting of colorectal cancer (CRC), gastric, esophageal, cervical, bladder, breast, head and neck, ovarian, melanoma, renal cell carcinoma (RCC), EC squamous cell, non-small cell lung carcinoma, mesothelioma, pancreatic, and prostate cancer.

15. The method of any one of claims 1-14 wherein the agent directed to human ICOS, or anti-ICOS antibody or antigen binding portion thereof, is administered as an intravenous (IV) infusion.

16. The method of any one of claims 1-15 wherein the agent directed to human OX40, or anti-PD1 antibody or antigen binding portion thereof or anti-PDL1 antibody or antigen binding portion thereof, is administered as an intravenous (IV) infusion.

17. The method of any one of claims 1-16 wherein the start of administration of the agent directed to human OX40 or the anti-OX40 antibody or antigen binding portion thereof, is initiated at a time point selected from 1 week, 2 weeks, 3 weeks, and 4 weeks after the start of the administration of the agent directed to human ICOS, or anti-ICOS antibody or antigen binding portion thereof.

18. The method of any one of claims 1-16 wherein the start of administration of the agent directed to human ICOS or the anti-ICOS antibody or antigen binding portion thereof, is initiated at a time point selected from 1 week, 2 weeks, 3 weeks, and 4 weeks after the start of the administration of the agent directed to human OX40, or anti-OX40 antibody or antigen binding portion thereof.

19. The method of any one of claims 1-18 wherein the agent directed to human ICOS, or the anti-ICOS antibody or antigen binding portion thereof, and the agent directed to human OX40 or the anti-OX40 antibody or antigen binding portion thereof, are administered to said human until said human shows disease progression or unacceptable toxicity.

20. An anti-ICOS antibody or antigen binding fragment thereof and an anti-OX40 antibody or antigen binding fragment thereof for sequential use in treating cancer in a human in need thereof, wherein the anti-ICOS antibody or antigen binding fragment thereof is administered prior to administration of the anti-OX40 antibody or antigen binding fragment thereof or wherein the anti-OX40 antibody or antigen binding fragment thereof is administered prior to administration of the anti-ICOS antibody or antigen binding fragment thereof.

21. An anti-ICOS antibody or antigen binding fragment thereof and an anti-OX40 antibody or antigen binding fragment thereof as claimed in claim 20, wherein the anti-OX40 antibody is an OX40 agonist.

22. An anti-ICOS antibody or antigen binding fragment thereof and an anti-OX40 antibody or antigen binding fragment thereof as claimed in claim 20 or 21, wherein the anti-OX40 antibody comprises a V_{H} domain comprising an amino acid sequence at least 90% identical to the amino acid sequence set forth in SEQ ID NO:5; and a V_{L} domain comprising an amino acid sequence at least 90% identical to the amino acid sequence as set forth in SEQ ID NO:11.

23. An anti-ICOS antibody or antigen binding fragment thereof and an anti-OX40 antibody or antigen binding fragment thereof as claimed in any one of claims 20-22, wherein the anti-OX40 antibody comprises a V_{H} domain comprising the amino acid sequence set forth in SEQ ID NO:5 and a V_{L} domain comprising the amino acid sequence as set forth in SEQ ID NO:11.

24. An anti-ICOS antibody or antigen binding fragment thereof and an anti-OX40 antibody or antigen binding fragment thereof as claimed in any one of claims 20-23, wherein the anti-ICOS antibody is an ICOS agonist.

25. An anti-ICOS antibody or antigen binding fragment thereof and an anti-OX40 antibody or antigen binding fragment thereof as claimed in any one of claims 20-24, wherein the anti-ICOS antibody comprises a V_{H} domain comprising an amino acid sequence at least 90% identical to the amino acid sequence set forth in SEQ ID NO:46; and a V_{L} domain comprising an amino acid sequence at least 90% identical to the amino acid sequence as set forth in SEQ ID NO:47.

26. An anti-ICOS antibody or antigen binding fragment thereof and an anti-OX40 antibody or antigen binding fragment thereof as claimed in any one of claims 20-25, wherein the anti-ICOS antibody comprises a V_{H} domain comprising the amino acid sequence set forth in SEQ ID NO:46 and a V_{L} domain comprising the amino acid sequence as set forth in SEQ ID NO:47.

27. An anti-ICOS antibody or antigen binding fragment thereof and an anti-OX40 antibody or antigen binding fragment thereof as claimed in any one of claims 20-26, wherein the anti-ICOS antibody is administered once every week, once every two weeks, once every three weeks, or once every four weeks.

28. An anti-ICOS antibody or antigen binding fragment thereof and an anti-OX40 antibody or antigen binding fragment thereof as claimed in any one of claims 20-27, wherein the anti-OX40 antibody is administered once every week, once every two weeks, once every three weeks, or once every four weeks.

29. An anti-ICOS antibody or antigen binding fragment thereof and an anti-OX40 antibody or antigen binding fragment thereof as claimed in any one of claims 20-28, wherein the cancer is selected from the group consisting of colorectal cancer (CRC), gastric, esophageal, cervical, bladder, breast, head and neck, ovarian, melanoma, renal cell carcinoma (RCC), EC squamous cell, non-small cell lung carcinoma, mesothelioma, pancreatic, and prostate cancer.

30. Use of an anti-ICOS antibody or antigen binding portion thereof and an anti-OX40 antibody or antigen binding portion thereof in the manufacture of a medicament for the treatment of cancer, wherein the anti-ICOS antibody or antigen binding portion thereof and the anti-OX40 antibody or antigen binding portion thereof are sequentially administered.

31. A polynucleotide encoding an anti-ICOS antibody or antigen binding portion thereof, wherein the anti-ICOS antibody or antigen binding portion thereof is sequentially administered to a cancer patient with an anti-OX40 antibody or antigen binding portion thereof.

32. A polynucleotide encoding an anti-OX40 antibody or antigen binding portion thereof, wherein the anti-OX40 antibody or antigen binding portion thereof is sequentially administered to a cancer patient with an anti-ICOS antibody or antigen binding portion thereof, and wherein administration of the anti-ICOS antibody or antigen binding portion thereof is followed by administration of the anti-OX40 antibody or antigen binding portion thereof.

33. A vector comprising the polynucleotide of any one of claims 31-32.

34. A host cell comprising the vector of claim 33.

35. A method of making an anti-ICOS antibody or antigen binding portion thereof, the method comprising a) culturing a host cell comprising the polynucleotide of claim 31 under suitable conditions to express the anti-ICOS antibody or antigen binding portion thereof; and b) isolating said anti-ICOS antibody or antigen binding portion thereof.

36. A method of making an anti-OX40 antibody or antigen binding portion thereof, the method comprising a) culturing a host cell comprising the polynucleotide of claim 32 under suitable conditions to express the anti-OX40 antibody or antigen binding portion thereof; and b) isolating said anti-OX40 antibody or antigen binding portion thereof.
